(19) United States
(12) Patent Application Publication
(10) Pub. No.: US 2009/0202920 A1
(43) Pub. Date: Aug. 13, 2009

(54) OPTICAL RECORDING COMPOSITION AND HOLOGRAPHIC RECORDING MEDIUM

(75) Inventor: Hiroyuki SUZUKI, Kanagawa (JP)

Correspondence Address: SUGHRUE MION, PLLC, 2100 PENNSYLVANIA AVENUE, N.W., SUITE 800, WASHINGTON, DC, 20037, US

(73) Assignee: FUJIFILM Corporation, Tokyo (JP)

(21) Appl. No.: 12/368,343

(22) Filed: Feb. 10, 2009

(30) Foreign Application Priority Data
Feb. 12, 2008 (JP) 2008-030179

(51) Int. Cl. G03F 7/00; C07D 417/06; C07D 277/08; C07D 263/54; G03F 7/004

(52) U.S. Cl. 430/2; 548/181; 548/146; 546/209; 546/198; 430/270.11

(57) ABSTRACT

The present invention provides an optical recording composition comprising at least one optical recording compound selected from the group consisting of cyanine compounds having specific structures and a holographic recording medium comprising a recording layer, wherein the recording layer comprises at least one optical recording compound selected from the group consisting of cyanine compounds having specific structures.

### CROSS-REFERENCE TO RELATED APPLICATIONS

This application claims the benefit of priority under 35 USC 119 to Japanese Patent Application No. 2008-30179 filed on Feb. 12, 2008, which is expressly incorporated herein by reference in its entirety.

### BACKGROUND OF THE INVENTION

1. Field of the Invention

The present invention relates to an optical recording composition comprising at least one optical recording compound in the form of a cyanine compound, and more particularly, to an optical recording composition suited to the manufacturing of a holographic recording medium permitting the writing of information, for example, with a 405 nm laser, particularly a volume holographic recording medium having a relatively thick recording layer. The present invention further relates to a holographic recording medium comprising a recording layer comprising the above optical recording compound.

2. Discussion of the Background

Holographic optical recording media based on the principle of the holograph have been developed. Recording of information on holographic optical recording media is carried out by superposing an informing light containing image information and a reference light in a recording layer comprised of a photosensitive composition to write an interference fringe thus formed in the recording layer. During the reproduction of information, a reference light is directed at a prescribed angle into the recording layer in which the information has been recorded, causing optical diffraction of the reference light by the interference fringe which has been formed, reproducing the informing light. For example, Published Japanese Translation of PCT International Application (TOKUHYO) No. 2005-502918 or English language family member US 2003/0087104 A1, which are expressly incorporated herein by reference in their entirety, discloses the use of a urethane matrix and a phenyl acrylate derivative in a holographic recording medium of the photopolymer type.

In recent years, volume holography, and, more particularly, digital volume holography, have been developed to practical levels for ultrahigh-density optical recording and have been garnering attention. Volume holography is a method of writing interference fringes three-dimensionally by also actively utilizing the direction of thickness of an optical recording medium. It is advantageous in that increasing the thickness permits greater diffraction efficiency and multiplexed recording increases the recording capacity. Digital volume holography is a computer-oriented holographic recording method in which the image data being recorded are limited to a binary digital pattern while employing a recording medium and recording system similar to those of volume holography. In digital volume holography, for example, image information such as an analog drawing is first digitized and then expanded into two-dimensional digital pattern information, which is recorded as image information. During reproduction, the digital pattern information is read and decoded to restore the original image information, which is displayed. Thus, even when the signal-to-noise (S/N) ratio deteriorates somewhat during reproduction, by conducting differential detection or conducting error correction by encoding the two-dimensional data, it is possible to reproduce the original data in an extremely faithful manner (see Japanese Unexamined Patent Publication (KOKAI) Heisei No. 11-311936 or English language family member US 2002/0114027 A1, which are expressly incorporated herein by reference in their entirety).

Greater recording capacity is being demanded of the above volume holographic optical recording media. For example, Japanese Unexamined Patent Publication (KOKAI) No. 2005-275158, US 2005/233246 A1, and Japanese Unexamined Patent Publication (KOKAI) No. 2007-272044, which are expressly incorporated herein by reference in their entirety, disclose recording media comprising recording monomers in the form of dye compounds for the purpose of increasing recording capacity.

The wavelength of the recording light has tended to become shorter in recent years to increase recording capacity. Specifically, the use of 405 nm recording lights has begun. However, in the recording media described in Japanese Unexamined Patent Publication (KOKAI) No. 2005-275158, US 2005/233246 A1, and Japanese Unexamined Patent Publication (KOKAI) No. 2007-272044, the transmittance at wavelengths around 400 nm of the media has decreased due to the use of dye compounds having substantial absorption in the visible light region. Thus, the above media make it difficult to conduct high-sensitivity recording with a recording light having a wavelength around 400 nm.

### SUMMARY OF THE INVENTION

An aspect of the present invention provides for an optical recording composition comprising an optical recording compound suited to high-sensitivity digital volume holography with large storage capacity in recording with short-wavelength light, and for a holographic recording medium permitting ultrahigh-density optical recording using the above optical recording composition.

As the result of extensive research, the present inventor discovered that it was possible to achieve a holographic recording medium capable of high-density and high-sensitivity recording with short-wavelength light by using the cyanine compounds denoted by general formulas (I-I) to (I-4) below. The present invention was devised on that basis.

An aspect of the present invention relates to an optical recording composition comprising at least one optical recording compound selected from the group consisting of compounds denoted by general formulas (I-1) to (I-4).

A further aspect of the present invention relates to a holographic recording medium comprising a recording layer, wherein the recording layer comprises at least one optical recording compound selected from the group consisting of compounds denoted by general formulas (I-1) to (I-4).

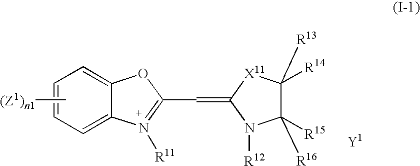

In general formula (I-1), each of R¹¹ and R¹² independently denotes an alkyl group, alkenyl group, aryl group, or heterocyclic group, each of R¹³, R¹⁴, R¹⁵, and R¹⁶ independently denotes a hydrogen atom or a substituent, X¹¹ denotes —S—, —O—, —NR¹¹¹—, or —CR¹¹²R¹¹³—, each of R¹¹¹, R¹¹², and R¹¹³ independently denotes a hydrogen atom, alkyl group, alkenyl group, aryl group, or heterocyclic group, R¹¹² and R¹¹³ may bond together to form a ring structure, Z¹ denotes a substituent, n1 denotes an integer ranging from 0 to 4, plural substituents denoted by Z¹ are identical to or different from each other when n1 denotes an integer of equal to or greater than 2, Y¹ denotes an ionic species that neutralizes an electric charge of the compound denoted by general formula (I-1), and at least one from among R¹¹, R¹², R¹³, R¹⁴, R¹⁵, R¹⁶, X¹¹, and Z¹ comprises at least one polymerizable group;

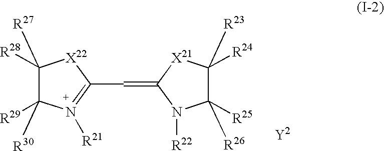

In general formula (I-2), each of R²¹ and R²² independently denotes an alkyl group, alkenyl group, aryl group, or heterocyclic group, each of R²³, R²⁴, R²⁵, R²⁶, R²⁷, R²⁸, R²⁹, and R³⁰ independently denotes a hydrogen atom or a substituent, each of X²¹ and X²² independently denotes —S—, —O—, —NR¹¹¹—, or —CR¹¹²R¹¹³—, each of R¹¹¹, R¹¹², and R¹¹³ independently denotes a hydrogen atom, alkyl group, alkenyl group, aryl group, or heterocyclic group, R¹¹² and R¹¹³ may bond together to form a ring structure, Y² denotes an ionic species that neutralizes an electric charge of the compound denoted by general formula (I-2), and at least one from among R²¹, R²², R²³, R²⁴, R²⁵, R²⁶, R²⁷, R²⁸, R²⁹, R³⁰, X²¹, and X²² comprises at least one polymerizable group;

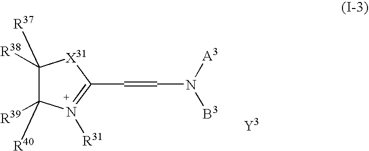

In general formula (I-3), R³¹ denotes an alkyl group, alkenyl group, aryl group, or heterocyclic group, each of R³⁷, R³⁸, R³⁹, and R⁴⁰ independently denotes a hydrogen atom or a substituent, X³¹ denotes —S—, —O—, —NR¹¹¹—, or —CR¹¹²R¹¹³, each of R¹¹¹, R¹¹², and R¹¹³ independently denotes a hydrogen atom, alkyl group, alkenyl group, aryl group, or heterocyclic group, R¹¹² and R¹¹³ may bond together to form a ring structure, each of A³ and B³ independently denotes a hydrogen atom, alkyl group, alkenyl group, aryl group, heterocyclic group, or acyl group, A³ and B³ may bond together to form a ring structure, Y³ denotes an ionic species that neutralizes an electric charge of the compound denoted by general formula (I-3), and at least one from among R³¹, R³⁷, R³⁸, R³⁹, R⁴⁰, X³¹, A³, and B³ comprises at least one polymerizable group; and

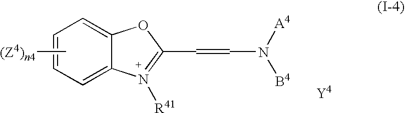

In general formula (I-4), R⁴¹ denotes an alkyl group, alkenyl group, aryl group, or heterocyclic group, each of A⁴ and B⁴ independently denotes a hydrogen atom, alkyl group, alkenyl group, aryl group, heterocyclic group, or acyl group, A⁴ and B⁴ may bond together to form a ring structure, Z⁴ denotes a substituent, n4 denotes an integer ranging from 0 to 4, plural substituents denoted by Z⁴ are identical to or different from each other when n4 denotes an integer of equal to or greater than 2, Y⁴ denotes an ionic species that neutralizes an electric charge of the compound denoted by general formula (I-4), and at least one from among R⁴¹, A⁴, B⁴, and Z⁴ comprises at least one polymerizable group.

The above optical recording compound may have a molar absorbance coefficient of equal to or lower than 200 mole-1-cm⁻¹ at a wavelength of 405 nm.

The above optical recording compound may have a maximum absorption wavelength of less than 405 nm.

The above polymerizable group may be a radical polymerizable group.

The above optical recording composition and/or the above recording layer may further comprise at least one photo-induced polymerization initiator.

The above photo-induced polymerization initiator may be a compound denoted by general formula (II):

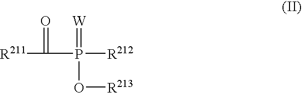

wherein, in general formula (II), each of R²¹¹, R²¹², and R²¹³ independently denotes an alkyl group, aryl group, or heterocyclic group, and W denotes an oxygen atom or sulfur atom.

The above optical recording composition and/or the above recording layer may further comprise at least one polyfunctional isocyanate and polyfunctional alcohol.

The above optical recording composition may be a holographic recording composition.

The optical recording compounds denoted by general formulas (I-1) to (I-4) are capable of high-sensitivity recording when conducting holographic recording employing a recording light source in the form of a laser having a central wavelength around 405 nm, specifically, 405±20 nm. The above compounds are also suited to digital volume holography, permitting the use of an inexpensive laser and a reduction in writing time.

The holographic recording medium of the present invention permits ultrahigh-density optical recording due to the presence of a holographic recording layer comprising one or more of the above compounds, and is optimal as a recording medium for volume holography, particularly digital volume holography.

Other exemplary embodiments and advantages of the present invention may be ascertained by reviewing the present disclosure and the accompanying drawings.

### BRIEF DESCRIPTION OF THE DRAWINGS

The present invention will be described in the following text by the exemplary, non-limiting embodiments shown in the figures, wherein:

FIG. 1 is a schematic cross-sectional view of an example of a holographic recording medium according to a first implementation embodiment.

FIG. 2 is a schematic cross-sectional view of an example of a holographic recording medium according to a second implementation embodiment.

FIG. 3 is a drawing descriptive of an example of an optical system permitting recording and reproducing of information on a holographic recording medium.

FIG. 4 is a block diagram showing an example of the overall configuration of a recording and reproducing device suited to use in recording and reproducing information on the holographic recording medium of the present invention.

FIG. 5 is a schematic of the optical system of a planar wave tester.

Explanations of symbols in the drawings are as follows:
1 Lower substrate
2 Reflective film
3 Servo pit pattern
4 Recording layer
5 Upper substrate
6 Filter layer
7 Second gap layer
8 First gap layer
12 Objective lens
13 Dichroic mirror
14 Detector
15 ¼ wavelength plate
16 Polarizing plate
17 Half mirror
20 Holographic recording medium
21 Holographic recording medium
22 Holographic recording medium
31 Pickup
81 Spindle
82 Spindle motor
83 Spindle servo circuit
84 Driving device
85 Detection circuit
86 Focus servo circuit
87 Tracking servo circuit
88 Slide servo circuit
89 Signal processing circuit
90 Controller
91 Operation element
100 Optical recording and reproducing device
A Entry and exit surface
FE Focus error signal
TE Tracking error signal
RF Reproduction signal

### DETAILED DESCRIPTIONS OF THE EMBODIMENTS

The following preferred specific embodiments are, therefore, to be construed as merely illustrative, and non-limiting to the remainder of the disclosure in any way whatsoever. In this regard, no attempt is made to show structural details of the present invention in more detail than is necessary for fundamental understanding of the present invention; the description taken with the drawings making apparent to those skilled in the art how several forms of the present invention may be embodied in practice.

### Optical Recording Composition

The optical recording composition of the present invention comprises at least one of cyanine compounds denoted by general formulas (I-1) to (I-4) below. The cyanine compounds denoted by general formulas (I-1) to (1-4) can be employed as a recording material in various recording systems in which information is recorded by irradiation with light. Among these, they are desirably employed as an optical recording compound such as a holographic recording compound, and are particularly suitable as a volume holographic recording compound. The holographic recording is a method of recording information by superposing an informing light containing information and a reference light in a recording layer to write an interference fringe thus formed in the recording layer. Volume holographic recording is a method of recording information in holographic recording in which a three-dimensional interference image is written in the recording layer. In the present invention, the term “holographic recording compound” refers to a compound that permits the recording of an interference fringe as refractive index modulation, either directly or indirectly, by irradiating light to record information. The compounds denoted by general formulas (I-1) to (I-4) can undergo a polymerization reaction, either directly or through the action of a photo-induced polymerization initiator, when irradiated with light, thereby permitting the recording of interference fringes as refractive index modulation.

The compounds denoted by general formulas (I-1) to (I-4) will be described in greater detail below.

### Compound Denoted by General Formula (I-1)

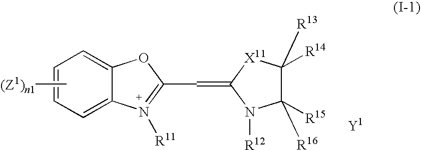

In general formula (I-1), each of R¹¹ and R¹² independently denotes an alkyl group, alkenyl group, aryl group, or heterocyclic group, with an alkyl group being desirable.

The alkyl group can be a linear, branched, or cyclic, substituted or unsubstituted alkyl group, desirably a substituted or unsubstituted linear or branched alkyl group having 1 to 30 carbon atoms (such as a methyl group, ethyl group, isopropyl group, n-propyl group, n-butyl group, t-butyl group, 2-pentyl group, n-hexyl group, n-octyl group, t-octyl group, 2-ethylhexyl group, 1,5-dimethylhexyl group, n-decyl group, n-dodecyl group, n-tetradecyl group, n-hexadecyl group, hydroxyethyl group, hydroxypropyl group, 2,3-dihydroxypropyl group, carboxymethyl group, carboxyethyl group, sodium sulfoethyl group, diethylaminoethyl group, diethylaminopropyl group, butoxypropyl group, ethoxyethoxyethyl group, or n-hexyloxypropyl group); or a substituted or unsubstituted cyclic alkyl group having 3 to 18 carbon atoms (such as a cyclopropyl group, cyclopentyl group, cyclohexyl group, cyclooctyl group, adamantyl group, or cyclododecyl group). It also includes a substituted or unsubstituted bicycloalkyl group having 5 to 30 carbon atoms, that is, a monovalent group in the form of bicycloalkane having 5 to 30 carbon atoms from which a hydrogen atom has been removed, such as a bicyclo[I-2,2]heptane-2-yl or bicyclo[2,2,2]octane-3-yl group, as well as a structure with a larger number of rings, such as a tricyclo structure.

The alkenyl group desirably denotes a substituted or unsubstituted alkenyl group having 2 to 30 carbon atoms, such as a vinyl group, allyl group, cycloalkenyl group (desirably a substituted or unsubstituted cycloalkenyl group having 3 to 30 carbon atoms, that is, a monovalent group in the form of a cycloalkene having 3 to 30 carbon atoms from which a hydrogen atom has been removed, such as 2-cyclopentene-1-yl group or 2-cyclohexene-1-yl group); or bicycloalkenyl group (substituted or unsubstituted bicycloalkenyl group, desirably a substituted or unsubstituted bicycloalkenyl group having 5 to 30 carbon atoms, that is, a monovalent group in the form of a bicycloalkene, having a single double bond, from which a hydrogen atom has been removed, such as a bicyclo[2,2,1]hept-2-ene-1-yl group or bicyclo[2,2,2]octo-2-ene-4-yl group).

The aryl group desirably denotes a substituted or unsubstituted aryl group having 6 to 30 carbon atoms, such as a phenyl group, p-tolyl group, naphthyl group, m-chlorophenyl group, or o-hexadecanoylaminophenyl group.

The heterocyclic group desirably denotes a substituted or unsubstituted, saturated or unsaturated heterocycle with a five to seven-membered ring comprising at least one from among a nitrogen atom, oxygen atom, or sulfur atom. It can be monocyclic or form a condensed ring with an aryl ring or heterocycle. The heterocyclic group is desirably five or six-membered, such as a pyrrolyl group, pyrrolidinyl group, pyridyl group, piperidyl group, piperazinyl group, imidazolyl group, pyrazolyl group, pyrazinyl group, pyrimidinyl group, triazinyl group, triazolyl group, tetrazolyl group, quinolyl group, isoquinolyl group, indolyl group, indazolyl group, benzoimidazolyl group, furyl group, pyranyl group, chromenyl group, thienyl group, oxazolyl group, oxadiazolyl group, thiazolyl group, thiadiazolyl group, benzooxazolyl group, benzothiazolyl group, morpholino group, or morpholinyl group.

Each of R¹³, R¹⁴, R⁵, and R¹⁶ independently denotes a halogen atom or a substituent.

Examples of the substituent are: a halogen atom (such as a fluorine, chlorine, bromine, or iodine atom), alkyl group, alkenyl group, alkynyl group, aryl group, heterocyclic group, acyl group, alkoxycarbonyl group, aryloxycarbonyl group, heterocyclic oxycarbonyl group, carbamoyl group, N-hydroxycarbamoyl group, N-acylcarbamoyl group, N-sulfonylcarbamoyl group, N-carbamoylcarbamoyl group, thiocarbamoyl group, N-sulfamoylcarbamoyl group, carbazoyl group, carboxy group (including a salt thereof), oxalyl group, oxamoyl group, cyano group, formyl group, hydroxy group, alkoxy group (including a group comprising repeating units of an ethyleneoxy or propyleneoxy group), aryloxy group, heterocyclic oxy group, acyloxy group, alkoxycarbonyloxy group, aryloxycarbonyloxy group, carbamoyloxy group, sulfonyloxy group, silyloxy group, nitro group, amino group, (alkyl, aryl, or heterocyclic) amino group, acylamino group, sulfonamide group, ureido group, thioureido group, N-hydroxyureido group, imido group, alkoxycarbonylamino group, aryloxycarbonylamino group, sulfamoylamino group, semicarbazide group, thiosemicarbazide group, hydrazine group, ammonio group, oxamoylamino group, N-(alkyl or aryl)sulfonylureido group, N-acylureido group, N-acylsulfamoylamino group, hydroxyamino group, quaternized nitrogen atom-containing heterocyclic group (such as a pyridinio group, imidazolio group, quinolinio group, or isoquinolinio group), isocyano group, imino group, alkylthio group, arylthio group, heterocyclic thio group, (alkyl, aryl, or heterocyclic) dithio group, (alkyl or aryl) sulfonyl group, (alkyl or aryl) sulfinyl group, sulfo group (including a salt thereof), sulfamoyl group, N-acylsulfamoyl group, N-sulfonylsulfamoyl group (including a salt thereof), or silyl group. In this context, the term “salt” means a salt with a cation, such as an alkali metal, alkaline earth metal, or heavy metal, or a salt with an organic cation, such as an ammonium ion or phosphonium ion. The substituent may be further substituted with one or more of the above substituents.

Each of R¹³, R¹⁴, R¹⁵, and R¹⁶ desirably denotes a hydrogen atom or an alkyl group, preferably a hydrogen atom.

X¹¹ denotes —S—, —O—, —NR¹¹¹—, or —CR¹¹²R¹¹³—. Each of R¹¹¹ to R¹¹³ independently denotes a hydrogen atom, alkyl group, alkenyl group, aryl group, or heterocyclic group. Examples of the alkyl groups, alkenyl groups, aryl groups, and heterocyclic groups denoted by R¹¹¹ to R¹¹³ are those of the alkyl groups, alkenyl groups, aryl groups, and heterocyclic groups denoted by R¹¹ and R¹² set forth above. R¹¹² and R¹¹³ may bond together to form a ring structure. Each of R¹¹¹ to R¹¹³ desirably denotes a hydrogen atom or an alkyl group, preferably an alkyl group.

X¹¹ desirably denotes —S— or —O—, and preferably denotes —S—.

Z¹ denotes a substituent. Specific examples of the substituent are those of the substituents denoted by R¹³, R¹⁴, R¹⁵, and R¹⁶ above. Z¹ desirably denotes a halogen atom, alkyl group, alkenyl group, aryl group, heterocyclic group, acyl group, alkoxycarbonyl group, aryloxycarbonyl group, carbamoyl group, alkoxy group, aryloxy group, acyloxy group, cyano group, acylamino group, or sulfonamide group; preferably denotes a halogen atom, alkyl group, alkoxycarbonyl group, carbamoyl group, alkoxy group, acyloxy group, cyano group, or acylamino group; and more preferably, denotes a halogen atom, alkoxy group, or acylamino group.

n1 denotes an integer ranging from 0 to 4, preferably 0 or 1. When n1 denotes an integer of equal to or greater than 2, plural substituents denoted by Z¹ may be identical to or different from each other.

Y¹ denotes an ionic species that neutralizes an electric charge of the compound denoted by general formula (I-1). Examples of such ionic species are: halide ions, OH⁻, SO₄²⁻, CO₃²⁻, NO₃⁻, H₂PO₃⁻, HPO₃²⁻, BF₄⁻, PF₆⁻, SbF₆⁻, ClO₄⁻, trifluoromethane sulfonate, methane sulfonate, benzene sulfonate, tosylate, and tetra(pentafluorophenyl)borate. Y¹ desirably denotes a halide ion, OH⁻SO₄²⁻, CO₃²⁻, NO₃⁻; H₂PO₃⁻, HPO₃²⁻, BF₄⁻, PF₆⁻, SbF₆⁻, ClO₄⁻, trifluoromethane sulfonate, methane sulfonate, benzene sulfonate, tosylate, or tetra(pentafluorophenyl)borate; preferably denotes Cl⁻, Br⁻, I⁻, SO₄²⁻, BF₄⁻, PF₆⁻, SbF₆⁻, ClO₄⁻, trifluoromethane sulfonate, methane sulfonate, benzene sulfonate, tosylate, or tetra(pentafluorophenyl)borate; and more preferably, denotes I⁻, BF₄⁻, PF₆⁻, SbF₆⁻, ClO₄⁻, trifluoromethane sulfonate, tosylate, or tetra(pentafluorophenyl)borate.

At least one from among R¹¹, R¹², R¹³, R¹⁴, R¹⁵, R¹⁶, X¹¹, and Z¹ comprises at least one polymerizable group. In the present invention, the term “polymerizable group” means a substituent capable of imparting a polymerization property to the compound denoted by general formula (I-1) so that it can be polymerized. More specifically, it is a substituent capable of causing a polymerizable component to be polymerized by irradiation with light, irradiation with radiation, heating, the use of a radical initiator, or the like. From the perspective of not undergoing reaction in the dark, a radical polymerizable group is desirable. A functional group capable of undergoing addition polymerization or condensation polymerization is preferred. The presence of a polymerizable group permits the direct or indirect recording of an interference fringe as refractive index modulation by irradiation with a recording light.

The above polymerizable group is desirably a functional group capable of undergoing addition polymerization. A polymerizable ethylenic unsaturated group or ring-opening polymerizable group is preferred as the polymerizable group, with a polymerizable ethylenic unsaturated group being of greater preference. Specifically, an acrylic group, methacrylic group, or styryl group is desirable; an acrylic group or methacrylic group is preferable; and an acrylic group is of greater preference. The substitution position of the polymerizable group is not specifically limited. In the compound denoted by general formula (I-1), one or more from among R¹¹, R¹², and Z¹ desirably comprises at least one polymerizable group. Examples of the polymerizable group are given below.

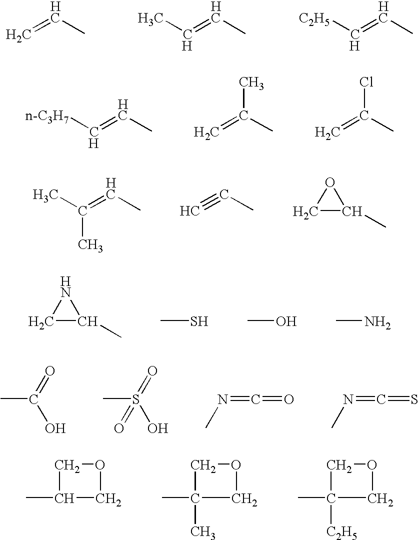

Formulas (M-1) to (M-6) below are examples of polymerizable ethylenic unsaturated groups.

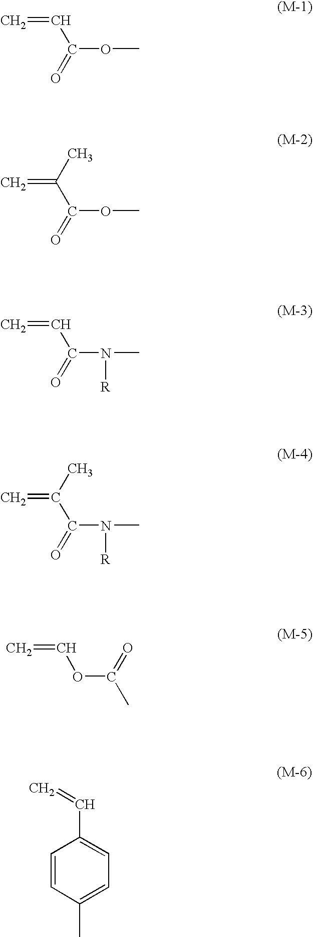

In formulas (M-3) to (M-4), R denotes a hydrogen atom or an alkyl group, desirably a hydrogen atom or a methyl group. Among formulas (M-1) to (M-6), an acrylic group denoted by formula (M-1), a methacrylic group denoted by formula (M-2), or a styryl group denoted by formula (M-6) is desirable, with an acrylic group denoted by formula (M-1) or a methacrylic group denoted by formula (M-2) being preferred.

### Compound Denoted by General Formula (I-2)

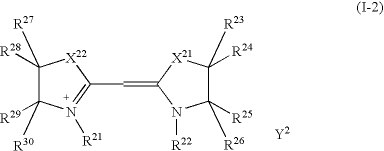

In general formula (I-2), each of R²¹ and R²² independently denotes an alkyl group, alkenyl group, aryl group, or heterocyclic group. The details thereof are as set forth above for R¹¹ and R¹² in general formula (I-1).

Each of R²³, R²⁴, R²⁵, R²⁶, R²⁷, R²⁸, R²⁹, and R³⁰ independently denotes a hydrogen atom or a substituent. The details thereof are as set forth above for R¹³ and the like in general formula (I-1).

Each of X²¹ and X²² independently denotes —S—, —O—, —NR¹¹¹—, or —CR¹¹²R¹¹³—. Each of R¹¹¹, R¹¹², and R¹¹³ independently denotes a hydrogen atom, alkyl group, alkenyl group, aryl group, or heterocyclic group. R¹¹² and R¹¹³ may bond together to form a ring structure. The details of X²¹ and X²² are as set forth above for X¹¹ in general formula (I-1). The details of R¹¹¹, R¹¹², and R¹¹³ are as set forth above.

Y² denotes an ionic species that neutralizes an electric charge of the compound denoted by general formula (I-2). The details thereof are as set forth above for Y¹ in general formula (I-1).

In general formula (I-2), at least one from among R²¹. R²², R²³, R²⁴, R²⁵, R²⁶, R²⁷, R²⁸, R²⁹, R³⁰, X²¹, and X²² comprises at least one polymerizable group. The details of the polymerizable group are as set forth above. Either R²¹ or R²² in general formula (I-2) desirably comprises at least one polymerizable group.

### Compound Denoted by General Formula (I-3)

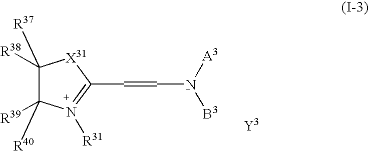

In general formula (I-3), R³¹ denotes an alkyl group, alkenyl group, aryl group, or heterocyclic group. The details thereof are as set forth above for R¹¹ in general formula (I-1).

Each of R³⁷, R³⁸, R³⁹, and R⁴⁰ independently denotes a hydrogen atom or a substituent. The details thereof are as set forth above for R¹³ and the like in general formula (I-1).

X³¹ denotes —S—, —O—, —NR¹¹¹—, or —CR¹¹²R¹¹³. Each of R¹¹¹, R¹¹², and R¹¹³ independently denotes a hydrogen atom, alkyl group, alkenyl group, aryl group, or heterocyclic group. R¹¹² and R¹¹³ may bond together to form a ring structure. The details of X³¹ are as set forth above for X¹¹ in general formula (I-1). The details of R¹¹¹, R¹¹², and R¹¹³ are as set forth above.

Each of A³ and B³ independently denotes a hydrogen atom, alkyl group, alkenyl group, aryl group, heterocyclic group, or acyl group. Examples of the alkyl groups, alkenyl groups, aryl groups, and heterocyclic groups denoted by A³ and B³ are those of the alkyl groups, alkenyl groups, aryl groups, and heterocyclic groups denoted by R¹¹ and R¹² in general formula (I-1) set forth above. Examples of the acyl groups denoted by A³ and B³ are substituted and unsubstituted acyl groups having 2 to 30 carbon atoms (such as acetyl groups, pivaloyl groups, 2-chloroacetyl groups, stearoyl groups, benzoyl groups, and p-n-octyloxyphenylcarbonyl groups). A³ and B³ may bond together to form a ring structure.

Y³ is an ionic species that neutralizes an electric charge of the compound denoted by general formula (I-3). The details thereof are as set forth above for Y¹ in general formula (I-1).

In general formula (I-3), at least one from among R³¹, R³⁷, R³⁸, R³⁹, R⁴⁰, X³¹, A³, and B³ comprises at least one polymerizable group. The polymerizable group is as set forth above. In general formula (I-3), it is desirable for one from among R³¹, R³², A³, and B³ to comprise at least one polymerizable group.

### Compound Denoted by General Formula (I-4)

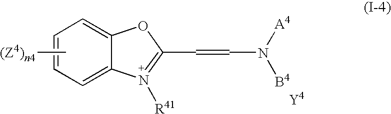

In general formula (I-4), R⁴¹ denotes an alkyl group, alkenyl group, aryl group, or heterocyclic group. The details thereof are as set forth above for R¹¹ in general formula (I-1).

Each of A⁴ and B⁴ independently denotes a hydrogen atom, alkyl group, alkenyl group, aryl group, heterocyclic group, or acyl group. A⁴ and B⁴ may bond together to form a ring structure. The details thereof are as set forth above for A³ and B³ in general formula (I-3).

Z⁴ denotes a substituent and n4 denotes an integer ranging from 0 to 4. When n4 denotes an integer of equal to or greater than 2, plural substituents denoted by Z⁴ may be identical to or different from each other. The details of Z⁴ are as set forth above for Z¹ in general formula (I-1). The details of n4 are as set forth above for n1 in general formula (I-1).

Y⁴ denotes an ionic species that neutralizes an electric charge of the compound denoted by general formula (I-4). The details thereof are as set forth above for Y¹ in general formula (I-1).

In general formula (I-4), at least one from among R⁴¹, A⁴, B⁴, and Z⁴ comprises at least one polymerizable group. The details of the polymerizable group are as set forth above. The polymerizable group can be contained by any of R⁴¹, A⁴, B⁴, and Z⁴.

Among those of the above-described substituents and functional groups that contain a hydrogen atom, the hydrogen atom can be removed and replaced with one of the above-described groups. Examples of such functional groups are: alkylcarbonylaminosulfonyl groups, arylcarbonylaminosulfonyl groups, alkylsulfonylaminocarbonyl groups, and arylsulfonylaminocarbonyl groups. Examples are: methylsulfonylaminocarbonyl groups, p-methylphenylsulfonylaminocarbonyl groups, acetylaminosulfonyl groups, and benzoylaminosulfonyl groups. In the present invention, the “number of carbon atoms” of a group means the number of carbon atoms of that group excluding substituents when present.

Specific examples of the compounds denoted by general formulas (I-1) to (1-4) are given below. However, the present invention is not limited thereto.

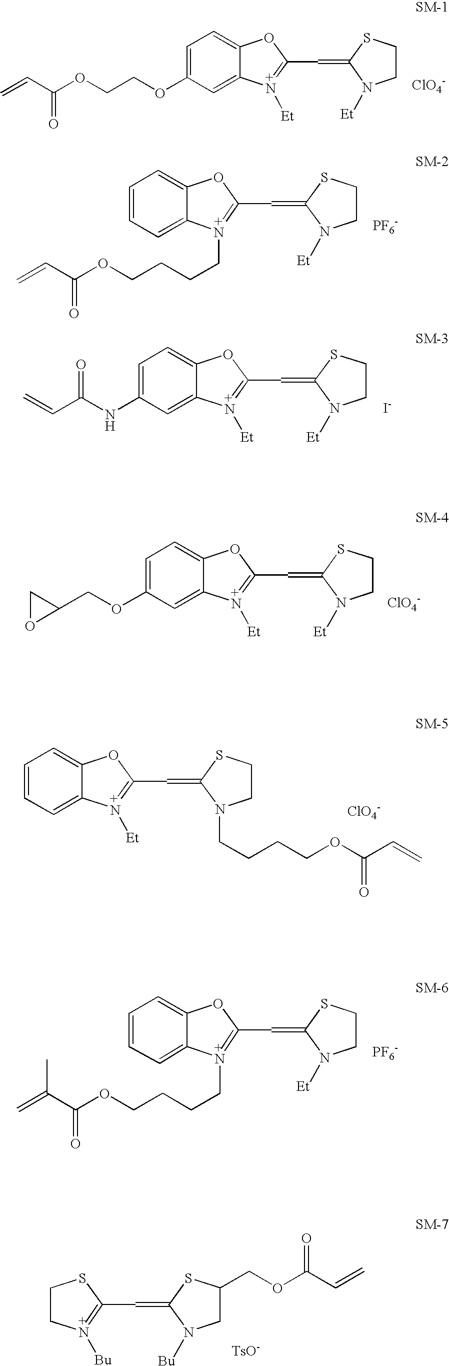

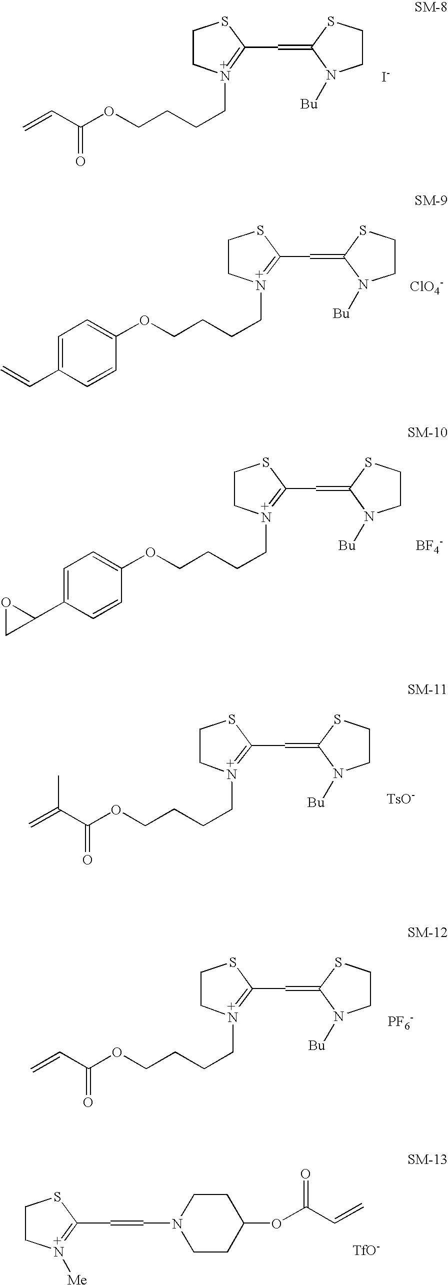

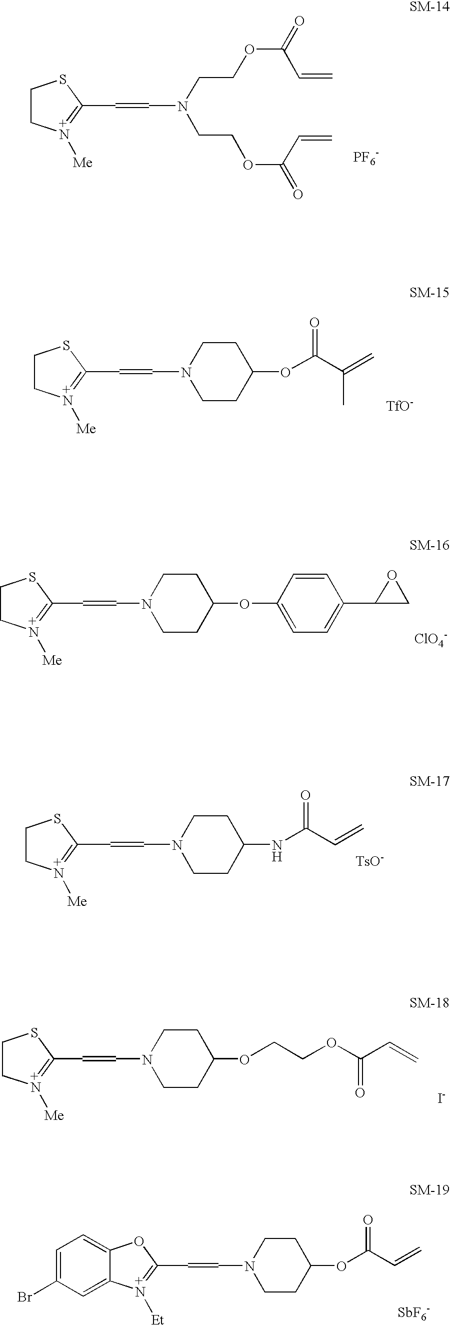

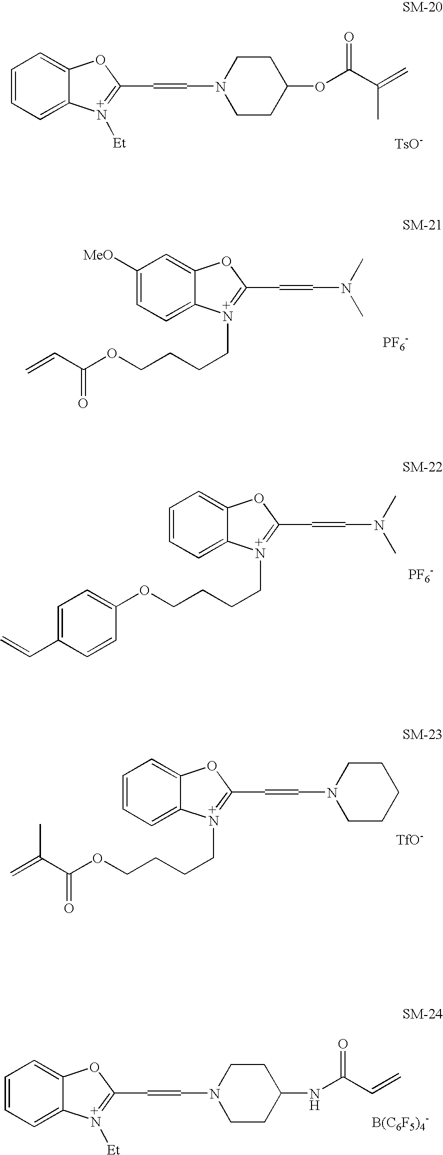

The compounds denoted by general formulas (I-1) to (I-4) can be synthesized by combinations of various known methods. The optimal synthesis method can be selected for a given compound. Specific examples of synthesis methods are given below.

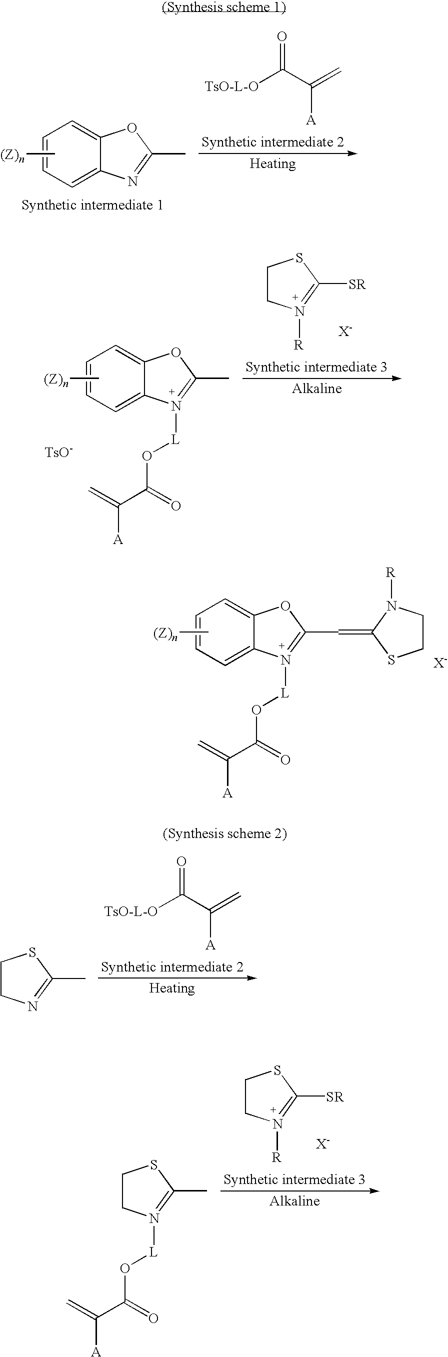

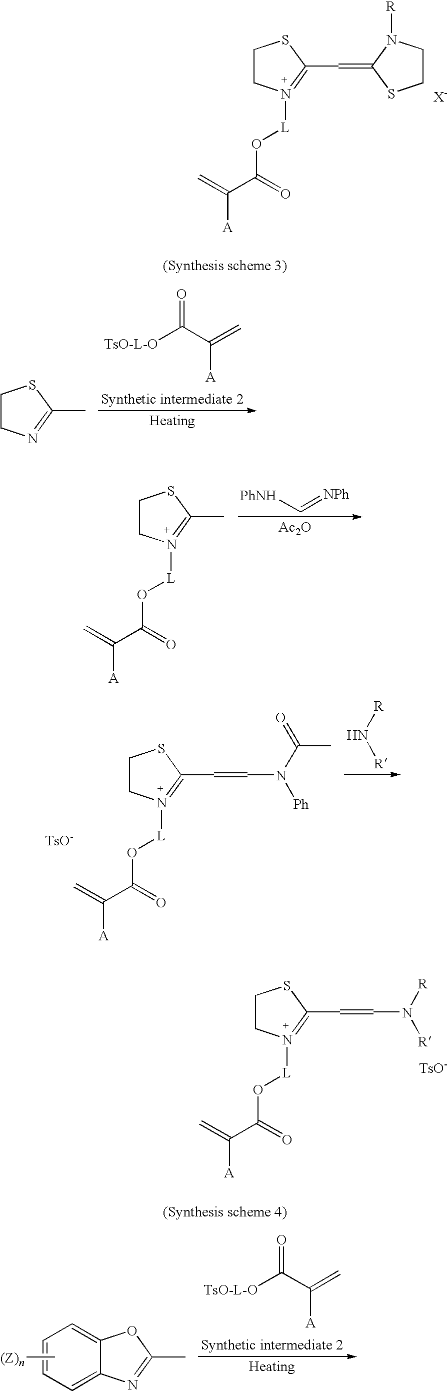

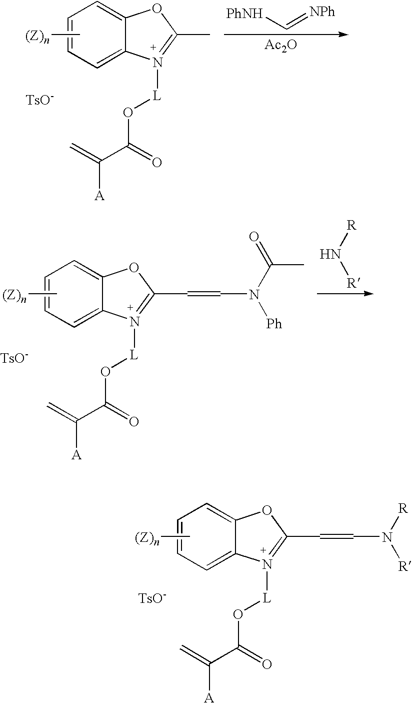

In the above synthesis schemes, Z denotes a substituent, n denotes an integer ranging from 0 to 4, L denotes a divalent linking group (for example, a group comprised of a combination of groups selected from among an alkylene group, arylene group, —O—, —S—, —C(═O)—, —SO₂⁻, or —NH— (where an alkyl group or the like can be substituted for the hydrogen atom in —NH—)). A denotes a hydrogen atom or alkyl group. R and R′ denote alkyl groups. X denotes an ionic species neutralizing the electric charge of the compound. Various commercial products can be employed for synthetic intermediate 1. Synthetic intermediate 2 can be synthesized by referring to Collect. Czech. Chem. Commun. 1983, No. 48, p. 3065; Chem. Commun. 1997, No. 5, p. 499, which are expressly incorporated herein by reference in their entirety; and the like, and selecting suitable starting materials.

Absorption at the recording wavelength in the compound employed as the recording compound in a holographic recording medium is desirably low so as to increase medium transmittance and achieve high sensitivity. The compounds denoted by general formulas (I-1) to (1-4) above can exhibit a molar absorbance coefficient ε of equal to or lower than 200 mole-1-cm⁻¹ at a wavelength of 405 nm, for example, and are thus suited to recording at a wavelength of around 400 nm. To achieve high recording capacity, it is desirable for the recording compound to have a great absorption at a wavelength shorter than the recording wavelength. The compounds denoted by general formulas (I-1) to (I-4) above can have a maximum absorption wavelength λmax of less than 405 nm, which is suitable for recording at a wavelength of around 400 nm. Specifically, the molar absorbance coefficient ε_{at 405 nm} at a wavelength of 405 nm of the compounds denoted by general formulas (I-1) to (I-4) is desirably equal to or lower than 200 mole-1-cm⁻¹, preferably falling within a range of 0to 100 mole-1-cm⁻¹. Further, the compounds denoted by general formula (I) desirably have a maximum absorption wavelength λmax of less than 405 nm, preferably falling within a range of 300 to 350 nm. The molar absorbance coefficient at λmax is desirably equal to or greater than 10,000 mole-1-cm⁻¹, preferably equal to or greater than 30,000 mole-1-cm⁻¹. The upper limit of the molar absorbance coefficient at λmax is not specifically limited. By way of example, it is about 200,000 mole-1-cm⁻¹.

The above absorption characteristics can be obtained from absorption spectra measured with a UV-visible light spectrophotometer for a solution obtained by dissolving the compound in a suitable solvent, such as methylene chloride.

The optical recording composition of the present invention comprises at least one of the compounds denoted by general formulas (I-1) to (I-4). Just one of the compounds denoted by general formulas (I-1) to (I-4) (also collectively referred to as “compounds denoted by general formula (I)”, hereinafter) can be employed, or two or more such compounds can be employed in combination. Of the compounds denoted by general formulas (I-1) to (I-4), the compounds denoted by general formulas (I-2) and (I-3) are desirable.

The content of the compounds denoted by general formula (I) in the optical recording composition of the present invention is not specifically limited and may be suitably selected based on the objective. A content of 1 to 50 weight percent is desirable, 1 to 30 weight percent is preferable, and 2 to 10 weight percent is of even greater preference. A content of equal to or less than 50 weight percent can readily ensure a stable interference image, and a content of equal to or greater than 1 weight percent can yield desirable properties from the perspective of diffraction efficiency.

### Photo-Induced Polymerization Initiator

The optical recording composition of the present invention is desirably employed as a holographic recording composition, and is particularly suited to use as a volume holographic recording composition. When employing the optical recording composition of the present invention as a holographic recording composition, the compounds denoted by general formula (I) can function as recording monomers. The optical recording composition of the present invention can comprise at least one photo-induced polymerization initiator in addition to the compounds denoted by general formula (I). The photo-induced polymerization initiator is not specifically limited, beyond that it be sensitive to the recording light. A material inducing a radical polymerization reaction, cationic ring-opening polymerization reaction, or the like when radiated with light can be employed. A photo-induced radical polymerization initiator is desirable from the perspective of polymerization reaction efficiency.

Examples of such photo-induced radical polymerization initiators are: 2,2′-bis(o-chlorophenyl)-4,4′-5,5′-tetraphenyl-1,1′-biimidazole, 2,4,6-tris(trichloromethyl)-1,3,5-triazine, 2,4-bis(trichloro-methyl)-6-(p-methoxyphenylvinyl)-1,3,5-triazine, diphenyliodoniumtetrafluoroborate, diphenyliodoniumhexafluorophosphate, 4,4′-di-t-butyldiphenyliodoniumtetrafluoroborate, 4-diethylaminophenylbenzenediazoniumhexafluorophosphate, benzoin, 2-hydroxy-2-methyl-1-phenylpropane-2-one, benzophenone, thioxanthone, 2,4,6-trimethylbenzoyl diphenylacyl phosphine oxide, triphenylbutylborate tetraethyl ammonium, diphenyl-4-phenylthiophenyl sulfonium hexafluorophosphate, 2,2-dimethoxy-1,2-diphenylethane-1-one, phenylglyoxylic acid methyl ester, 2-methyl-1-[4-(methylthio)phenyl]-2-morpholinopropane-1-one, bis(2,4,6-trimethylbenzoyl)phenylphosphine oxide, 2,4,6-trimethylbenzoyl diphenyl phosphine oxide, 1,2-octanedione, 1-[4-(phenylthio)-2-(0-benzoyloxime)], and bis(η5-2,4-cyclopentadien-1-yl)bis[2,6-difluoro-3-(1H-pyrrol-1-yl)phenyltitanium]. These may be employed singly or in combinations of two or more. A sensitizing dye, described further below, may also be employed in combination based on the wavelength of the light being irradiated.

Among photo-induced radical polymerization initiators, the suitable photo-induced radical polymerization initiator may be a compound denoted by general formula (II).

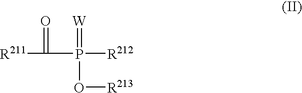

In general formula (II), each of R²¹¹, R²¹² and R²¹³ independently denotes an alkyl group, aryl group or heterocyclic group, and W denotes an oxygen atom or sulfur atom.

The compound denoted by general formula (II) will be described in detail below.

In general formula (II), each of R²¹¹, R²¹² and R²¹³ independently denotes an alkyl group, aryl group, or heterocyclic group.

The alkyl groups denoted by R²¹¹, R²¹², and R²¹³ can be linear or branched, and substituted or unsubstituted. They desirably have 1 to 30 carbon atoms, preferably 1 to 20 carbon atoms.

Examples of the alkyl groups denoted by R²¹¹, R²¹² and R²¹³ are: methyl groups, ethyl groups, normal propyl groups, isopropyl groups, normal butyl groups, isobutyl groups, tertiary butyl groups, pentyl groups, cyclopentyl groups, hexyl groups, cyclohexyl groups, heptyl groups, octyl groups, tertiary octyl groups, 2-ethylhexyl groups, decyl groups, dodecyl groups, octadecyl groups, 2,3-dibromopropyl groups, adamantyl groups, benzyl groups, and 4-bromobenzyl groups. These may be further substituted. Of these, tertiary butyl groups are greatly preferred from the perspective of stability in the presence of nucleophilic compounds, such as water and alcohol.

The aryl groups denoted by R²¹¹, R²¹², and R²¹³ in general formula (II) can be substituted or unsubstituted. They desirably comprise 6 to 30 carbon atoms, preferably 6 to 20 carbon atoms. Specific examples of these aryl groups are: phenyl groups, naphthyl groups, and anthranyl groups. These may be further substituted. Of these, R²¹¹ desirably denotes an aryl group in which an alkyl group, aryl group, alkoxy group, or halogen group is present at position 2, and preferably denotes an aryl group in which an alkyl group, aryl group, alkoxy group, or halogen group is present at positions 2 and 6. For example, R²¹¹ desirably denotes a 2-methylphenyl group, 2,4,6-trimethylphenyl group, 2,6-dichlorophenyl group, 2,6-dimethoxyphenyl group, or 2,6-trifluoromethylphenyl group, and preferably denotes a 2,4,6-trimethylphenyl group, 2,6-dichlorophenyl group, or 2,6-dimethoxyphenyl group. The presence of the above substituents at position 2, or at positions 2 and 6, is desirable to enhance stability in the presence of nucleophilic compounds, such as water and alcohols, as described in, for example, Jacobi, M., Henne, A. Polymers Paint Colour Journal 1985, 175, 636, which is expressly incorporated herein by reference in its entirety. Details of desirable examples of alkyl groups and aryl groups employed as the above substituents are identical to those set forth for the alkyl groups and aryl groups denoted by R²¹¹, R²¹², and R²¹³ above.

The heterocyclic groups denoted by R²¹¹, R²²¹, and R²¹³ in general formula (II) are desirably four to eight-membered rings, preferably four to six-membered rings, and more preferably, five or six-membered rings. Specific examples are: pyridine rings, piperazine rings, thiophene rings, pyrrole rings, imidazole rings, oxazole rings, and thiazole rings. They may be further substituted. Of these hetero rings, pyridine rings are particularly desirable.

When the groups denoted by R²¹¹, R²¹², and R²¹³ in general formula (II) comprise one or more substituents, examples of the substituents are: halogen groups, alkyl groups, alkenyl groups, alkoxy groups, aryloxy groups, alkylthio groups, alkoxycarbonyl groups, aryloxycarbonyl groups, amino groups, acyl groups, alkylaminocarbonyl groups, arylaminocarbonyl groups, sulfonamide groups, cyano groups, carboxy groups, hydroxyl groups, and sulfonic acid groups. Of these, halogen groups, alkoxy groups, and alkylthio groups are particularly desirable. When R²¹¹ denotes an aryl group as set forth above, the above substituents are desirably present at position 2, or positions 2 and 6, on the aryl group.

In general formula (II), W denotes an oxygen atom or a sulfur atom, desirably an oxygen atom.

Examples of desirable compounds denoted by general formula (II) are compounds in which R²¹¹ denotes an aryl group with an alkyl group, aryl group, alkoxy group, or halogen group present at position 2, R²¹² denotes an aryl group, R²¹³ denotes an alkyl group, and W denotes an oxygen atom or a sulfur atom. Examples of preferred compounds are compounds in which R²¹¹ denotes an aryl group with an alkyl group, aryl group, alkoxy group, or halogen group present at positions 2 and 6, R²¹² denotes an aryl group, R²¹³ denotes an alkyl group, and W denotes an oxygen atom. Examples of compounds of greater preference are compounds in which R²¹¹ denotes a 2,6-dimethoxybenzoyl group or 2,6-dichlorobenzoyl group, R²¹² denotes a phenyl group, R²¹³ denotes an ethyl group or isopropyl group, and W denotes an oxygen atom.

Specific examples of the phosphorus compound denoted by general formula (II) are given below. However, the present invention is not limited to these specific examples.

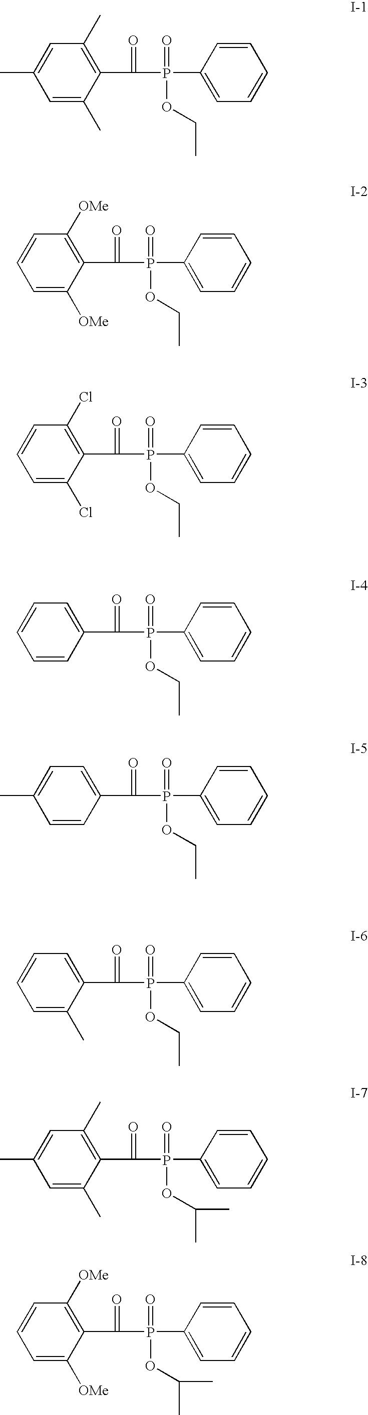

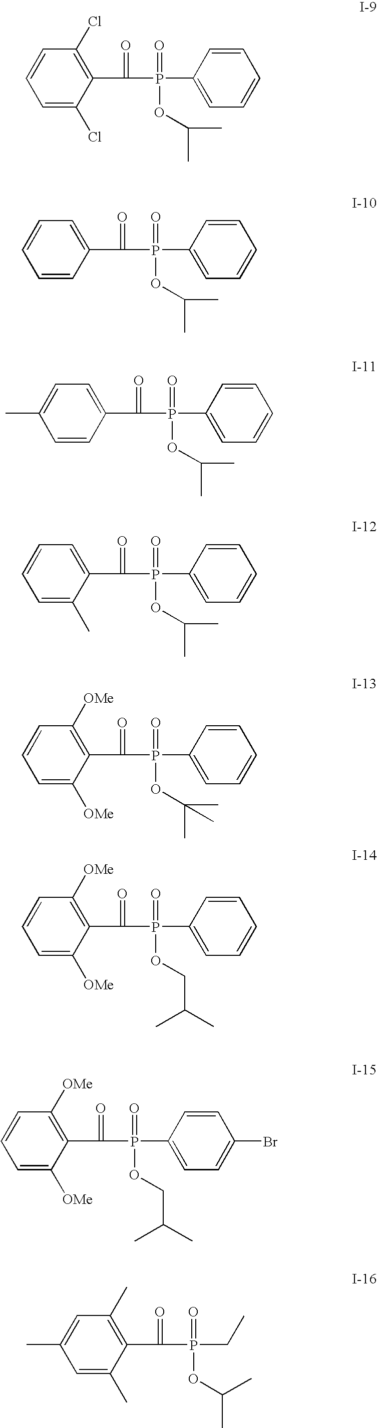

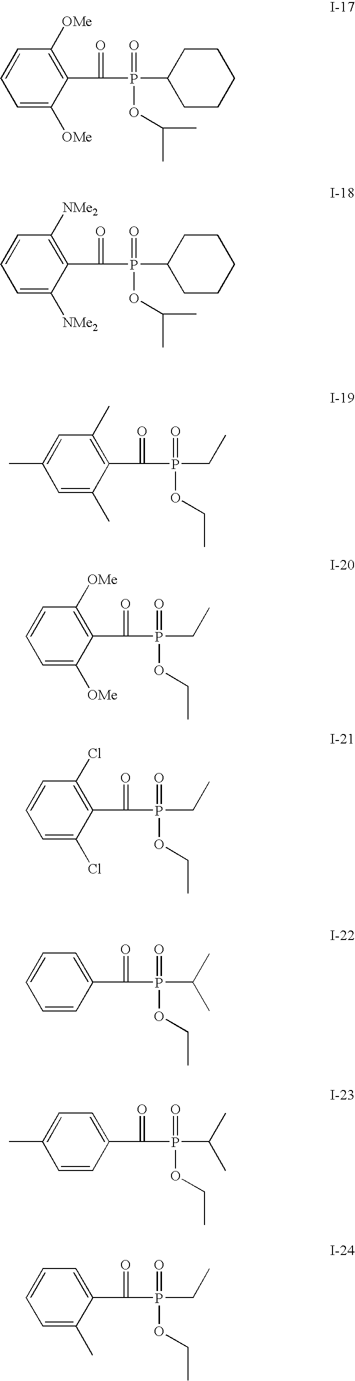

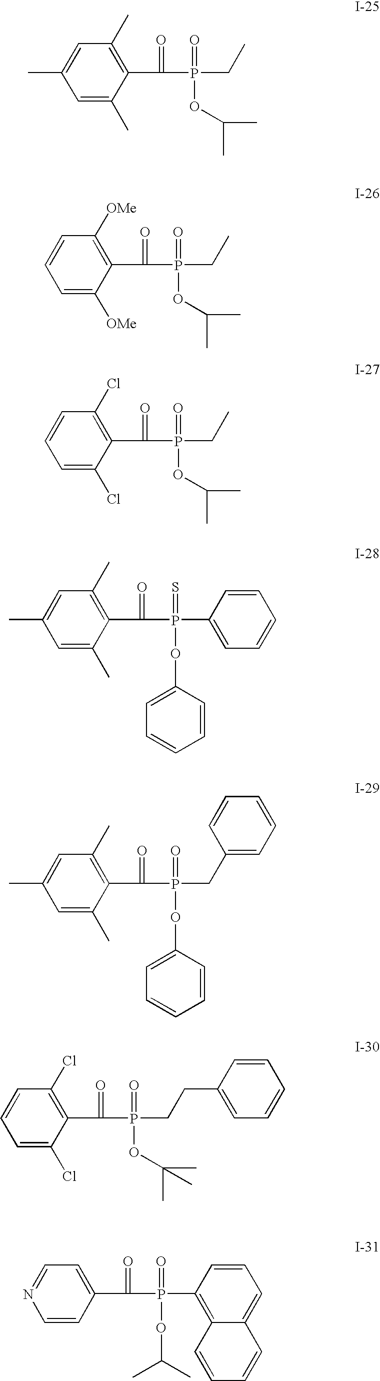

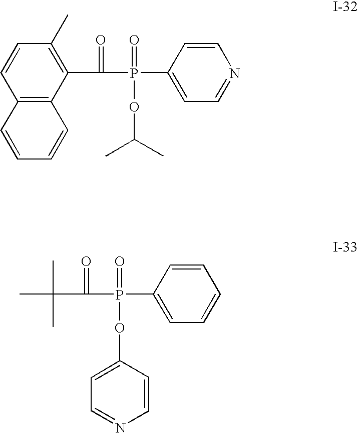

A method of synthesizing the above-described compound denoted by general formula (II) is described in detail in, for example, DE2830927A1, which is expressly incorporated herein by reference in its entirety. Examples described further below can also be referred to for synthesis methods.

Examples of cationic ring-opening photopolymerization initiators are 2,4,6-tris(trichloromethyl)-1,3,5-triazine, 2,4-bis(trichloromethyl)-6-(p-methoxyphenylvinyl)-1,3,5-triazine, diphenyliodonium tetrafluoroborate, 4,4′-di-t-butyldiphenyliodonium tetrafluoroborate, 4-diethylaminophenylbenzenediazonium hexafluorophosphate, and diphenyl-4-phenylthiophenylsulfonium hexafluorophosphate. These may be employed singly or in combinations of two or more. Sensitizing dyes, described further below, may be employed in combination in a manner in conformity with the wavelength of the light that is irradiated.

The content of the photo-induced polymerization initiator in the optical recording composition of the present invention is desirably 0.01 to 5 weight percent, preferably 1 to 3 weight percent. The content of equal to or greater than 0.01 weight percent can ensure an interference image of good sensitivity. The content of equal to or lower than 5 weight percent can permit the formation of a recording layer having adequate transmittance of the recording light and exhibiting good recording sensitivity.

The compounds denoted by general formula (I) may be monofunctional monomers having a single polymerizable group, or may be polyfunctional monomers having two or more such groups per molecule. In the optical recording composition of the present invention, just compounds denoted by general formula (I) may be incorporated as recording compounds, or another polymerizable monomer may be incorporated along with the compounds denoted by general formula (I). When another polymerizable monomer is incorporated along with the compounds denoted by general formula (I), the quantity of the additional polymerizable monomer employed is desirably equal to or lower than 50 weight percent of the total polymerizable monomer.

Examples of additional monomers in the form of radical polymerizable monomers are: acryloylmorpholine, phenoxyethyl acrylate, isobornyl acrylate, 2-hydroxypropyl acrylate, 2-ethylhexyl acrylate, 1,6-hexanediol diacrylate, tripropyleneglycol diacrylate, neopentylglycol PO-modified diacrylate, 1,9-nonanediol diacrylate, hydroxypivalic acid neopentylglycol diacrylate, EO-modified bisphenol A diacrylate, polyethlyleneglycol diacrylate, pentaerythritol triacrylate, pentaerythritol tetraacrylate, pentaerythritol hexaacrylate, EO-modified glycerol triacrylate, trimethylolpropane triacrylate, EO-modified trimethylolpropane triacrylate, 2-naphtho-1-oxyethyl acrylate, 2-carbazoyl-9-ylethyl acrylate, (trimethylsilyloxy)dimethylsilylpropyl acrylate, vinyl-1-naphthoate, 2,4,6-tribromophenyl acrylate, pentabromoacrylate, phenylthioethyl acrylate, tetrahydrofurfuryl acrylate, bisphenoxyethanolfluorene diacrylate, styrene, p-chlorostyrene, N-vinylcarbazole, and N-vinylpyrrolidone. Of these, phenoxyethyl acrylate, 2,4,6-tribromophenyl acrylate, pentabromoacrylate, and bisphenoxyethanolfluorene diacrylate are desirable, and 2,4,6-tribromophenyl acrylate and bisphenoxyethanolfluorene diacrylate are preferred.

Examples of additional monomers in the form of cationic polymerizable monomers are: 2,3-epoxy-1-propane, 3,4-epoxy-1-butane, 1,6-hexanediol monoglycidyl ether, glycerol diglycidyl ether, glycerol propoxylate diglycidyl ether, glycidyl 4-hydroxyphenyl ether, glycidyl phenyl ether, 1,2-epoxyethylbenzene, bisphenol A diglycidyl ether, pentaerythritol tetra(3-ethyl-3-oxycetanylmethyl)ether, 3-ethylene carbonate, propylene carbonate, and γ-butyrolactone.

### Matrix

The recording layer of an optical recording medium normally comprises a polymer to hold the photopolymerization initiator and monomers related to the recording and storage, known as a matrix. The matrix can be employed for achieving enhanced coating properties, coating strength, and hologram recording characteristics. The optical recording composition of the present invention can comprise curing compounds in the form of a matrix binder and/or matrix forming components (matrix precursors). A method of forming the matrix by, for example, coating a composition containing the matrix precursor on the surface of a substrate and then curing it is desirable because it permits the formation of the recording layer without the use of, or using only a small quantity of, solvent. Thermosetting compounds and light-curing compounds employing catalysts and the like that cure when irradiated with light may be employed as these curing compounds. Thermosetting compounds are desirable from the perspective of recording characteristics.

The thermosetting compound suitable for use in the optical recording composition of the present invention is not specifically limited. The matrix contained in the recording layer may be suitably selected based on the objective. Examples are urethane resins formed from isocyanate compounds and alcohol compounds; epoxy compounds formed from oxysilane compounds; melamine compounds; formalin compounds; ester compounds of unsaturated acids such as (meth)acrylic acid and itaconic acid; and polymers obtained by polymerizing amide compounds.

Of these, polyurethane matrices formed from isocyanate compounds and alcohol compounds are preferable. From the perspective of recording retention properties, three-dimensional polyurethane matrices formed from polyfunctional isocyanates and polyfunctional alcohols are particularly preferred.

The details of polyfunctional isocyanates and polyfunctional alcohols capable of forming polyurethane matrices are described below.

Examples of the polyfunctional isocyanates are: biscyclohexylmethane diisocyanate, hexamethylene diisocyanate, phenylene-1,3-diisocyanate, phenylene-1,4-diisocyanate, 1-methoxyphenylene-2,4-diisocyanate, 1-methylphenylene-2,4-diisocyanate, 2,4-tolylene diisocyanate, 2,6-tolylene diisocyanate, 1,3-xylylene diisocyanate, 1,4-xylylene diisocyanate, biphenylene-4,4′-diisocyanate, 3,3′-dimethoxybiphenylene-4,4′-diisocyanate, 3,3′-dimethylbiphenylene-4,4′-diisocyanate, diphenyhnethane-2,4′-diisocyanate, diphenylmethane-4,4′-diisocyanate, 3,3′-dimethoxydiphenylmethane-4,4′-diisocyanate, 3,3′-dimethyldiphenylmethane-4,4′-diisocyanate, naphthylene-1,5-diisocyanate, cyclobutylene-1,3-diisocyanate, cyclopentylene-1,3-diisocyanate, cyclohexylene-1,3-diisocyanate, cyclohexylene-1,4-diisocyanate, 1-methylcyclohexylene-2,4-diisocyanate, 1-methylcyclohexylene-2,6-diisocyanate, 1-isocyanate-3,3,5-trimethyl-5-isocyanate methylcyclohexane, cyclohexane-1,3-bis(methylisocyanate), cyclohexane-1,4-bis(methylisocyanate), isophorone diisocyanate, dicyclohexylmethane-2,4′-diisocyanate, dicyclohexylmethane-4,4′-diisocyanate, ethylene diisocyanate, tetramethylene-1,4-diisocyanate, hexamethylene-1,6-diisocyanate, dodecamethylene-1,12-diisocyanate, phenyl-1,3,5-triisocyanate, diphenylmethane-2,4,4′-triisocyanate, diphenylmethane-2,5,4′-triisocyanate, triphenylmethane-2,4′,4″-triisocyanate, triphenylmethane-4,4′,4″-triisocyanate, diphenylmethane-2,4,2′,4′-tetraisocyanate, diphenylmethane-2,5,2′,5′-tetraisocyanate, cyclohexane-1,3,5-triisocyanate, cyclohexane-1,3,5-tris(methylisocyanate), 3,5-dimethylcyclohexane-1,3,5-tris(methylisocyanate), 1,3,5-trimethylcyclohexane-1,3,5-tris(methylisocyanate), dicyclohexylmethane-2,4,2′-triisocyanate, dicyclohexylmethane-2,4,4′-triisocyanate lysine isocyanate methyl ester, and prepolymers having isocyanates on both ends obtained by reacting a stoichiometrically excess quantity of one or more of these organic isocyanate compounds with a polyfunctional active hydrogen-containing compound. Of these, biscyclohexylmethane diisocyanate and hexamethylene diisocyanate are preferred. They may be employed singly or in combinations of two or more.

The polyfunctional alcohols may be in the form of a single polyfunctional alcohol, or in the form of a mixture with two or more polyfunctional alcohols. Examples of these polyfunctional alcohols are: glycols such as ethylene glycol, triethylene glycol, diethylene glycol, polyethylene glycol, propylene glycol, polypropylene glycol, and neopentyl glycol; diols such as butanediol, pentanediol, hexanediol, heptanediol, and tetramethylene glycol; bisphenols; compounds in the form of these polyfunctional alcohols modified by polyethyleneoxy chains or polypropyleneoxy chains; and compounds in the form of these polyfunctional alcohols modified by polyethyleneoxy chains or polypropyleneoxy chains, such as glycerin, trimethylolpropane, butanetriol, pentanetriol, hexanetriol, decanetriol, and other triols.

The content of the above-described matrix-forming components (or matrix) in the optical recording composition of the present invention is desirably 10 to 95 weight percent, preferably 35 to 90 weight percent. When the content is equal to or greater than 10 weight percent, stable interference images can be readily achieved. At equal to or less than 95 weight percent, desirable properties can be obtained from the perspective of diffraction efficiency.

### Other Components

Polymerization inhibitors and oxidation inhibitors may be added to the optical recording composition of the present invention to improve the storage stability of the optical recording composition, as needed.

Examples of polymerization inhibitors and oxidation inhibitors are: hydroquinone, p-benzoquinone, hydroquinone monomethyl ether, 2,6-ditert-butyl-p-cresol, 2,2′-methylenebis(4-methyl-6-tert-butylphenol), triphenylphosphite, trisnonylphenylphoshite, phenothiazine, and N-isopropyl-N′-phenyl-p-phenylenediamine.

The quantity of polymerization inhibitor or oxidation inhibitor added is preferably equal to or less than 3 weight percent of the total quantity of recording monomer. When the quantity added exceeds 3 weight percent, polymerization may slow down, and in extreme cases, ceases.

As needed, a sensitizing dye may be added to the optical recording composition of the present invention. Known compounds such as those described in “Research Disclosure, Vol. 200, 1980, December, Item 20036” and “Sensitizers” (pp. 160-163, Kodansha, ed. by K. Tokumaru and M. Okawara, 1987), which are expressly incorporated herein by reference in their entirety, and the like may be employed as sensitizing dyes.

Specific examples of sensitizing dyes are: 3-ketocoumarin compounds described in Japanese Unexamined Patent Publication (KOKAI) Showa No. 58-15603; thiopyrilium salt described in Japanese Unexamined Patent Publication (KOKAI) Showa No. 58-40302; naphthothiazole merocyanine compounds described in Japanese Examined Patent Publications (KOKOKU) Showa Nos. 59-28328 and 60-53300; and merocyanme compounds described in Japanese Examined Patent Publications (KOKOKU) Showa Nos. 61-9621 and 62-3842 and Japanese Unexamined Patent Publications (KOKAI) Showa Nos. 59-89303 and 60-60104, which are expressly incorporated herein by reference in their entirety.

Further examples are the dyes described in “The Chemistry of Functional Dyes” (1981, CMC Press, pp. 393-416) and “Coloring Materials” (60 [4] 212-224 (1987)), which are expressly incorporated herein by reference in their entirety. Specific examples are cationic methine dyes, cationic carbonium dyes, cationic quinoneimine dyes, cationic indoline dyes, and cationic styryl dyes.

Further, keto dyes such as coumarin (including ketocoumarin and sulfonocoumarin) dyes, merostyryl dyes, oxonol dyes, and hemioxonol dyes; nonketo dyes such as nonketo polymethine dyes, triarylmethane dyes, xanthene dyes, anthracene dyes, rhodamine dyes, acrylidine dyes, aniline dyes, and azo dyes; nonketo polymethine dyes such as azomethine dyes, cyanine dyes, carbocyanine dyes, dicarbocyanine dyes, tricarbocyanine dyes, hemicyanine dyes, and styryl dyes; and quinone imine dyes such as azine dyes, oxazine dyes, thiazine dyes, quinoline dyes, and thiazole dyes are included among the spectral sensitizing dyes.

These sensitizing dyes may be employed singly or in combinations of two or more.

A photo-heat converting material can be incorporated into the optical recording composition of the present invention for enhancing the sensitivity of the recording layer formed with the optical recording composition.

The photo-heat converting material is not specifically limited, and may be suitably selected based on the functions and properties desired. For example, for convenience during addition to the recording layer with the photopolymer and so as not to scatter incident light, an organic dye or pigment is desirable. From the perspectives of not absorbing and not scattering light from the light source employed in recording, infrared radiation-absorbing dyes are desirable.

Such infrared radiation-absorbing dyes are not specifically limited, and may be suitably selected based on the objective. However, cationic dyes, complex-forming dyes, quinone-based neutral dyes, and the like are suitable. The maximum absorption wavelength of the infrared radiation-absorbing dye preferably falls within a range of 600 to 1,000 nm, more preferably a range of 700 to 900 nm.

The content of infrared radiation-absorbing dye in the optical recording composition of the present invention can be determined based on the absorbance at the wavelength of maximum absorbance in the infrared region in the recording medium formed with the optical recording composition of the present invention. This absorbance preferably falls within a range of 0.1 to 2.5, more preferably a range of 0.2 to 2.0.

The optical recording composition of the present invention can be employed as various holographic recording compositions capable of recording information when irradiated with a light containing information. In particular, it is suited to use as a volume holographic recording composition. A recording layer can be formed by coating the optical recording composition of the present invention on a substrate, for example. When the optical recording composition of the present invention contains a thermosetting compound such as those set forth above, a matrix can be formed by promoting the curing reaction by heating following coating. The heating conditions can be determined based on the thermosetting resin employed. The recording layer can be formed by casting when the viscosity of the optical recording composition is adequately low. When the viscosity is so high that casting is difficult, a dispenser can be employed to spread a recording layer on a lower substrate, and an upper substrate pressed onto the recording layer so as to cover it and spread it over the entire surface, thereby forming a recording medium.

### Holographic Recording Medium

The holographic recording medium of the present invention comprises a recording layer comprising at least one compound denoted by general formula (I). The recording layer can be formed with the optical recording composition of the present invention. For example, the recording layer comprised of the optical recording composition of the present invention can be formed by the above-described method.

The recording layer of the holographic recording composition of the present invention comprises one or more of the compounds denoted by general formula (I). Since the compounds denoted by general formula (I) have absorption characteristics that are suited to recording by irradiation with light of short wavelength, it is possible to form a holographic recording medium permitting highly sensitive, high-density recording in the short wavelength recording region. In the same manner as for the content in the optical recording composition of the present invention set forth above, the content of the compound denoted by general formula (I) in the recording layer is desirably 1 to 50 weight percent, preferably 1 to 30 weight percent, and more preferably, 2 to 10 weight percent. A content of equal to or lower than 50 weight percent can readily ensure a stable interference image, and a content of equal to or greater than 1 weight percent can yield desirable properties from the perspective of diffraction efficiency. The details of the various components in the recording layer of the holographic recording medium of the present invention are as set forth above for the optical recording composition of the present invention.

The holographic recording medium of the present invention is particularly suited to use as a holographic recording medium with a light source having a wavelength of around 400 nm. Since the holographic recording medium employs an incident diffraction light as a signal light, transmittance of the recording and reproducing lights is desirably high. For example, for a recording wavelength of 405 nm and a recording layer 500 μm in thickness, the addition of a polymerizable compound with a molecular weight of 400 in an amount of 10 weight percent relative to the matrix yields a concentration of about 0.018 mol/L. Considering the case where an initiator having a molar absorbance coefficient of about 80 mole-1-cm⁻¹ at 405 nm is added in a proportion of 15 molar percent relative to the quantity of the polymerizable compound, the transmittance of the recording layer is less than 60 percent when the molar absorbance coefficient of the polymerizable compound is equal to or greater than 200 mole-1-cm⁻¹. Since it is desirable for the transmittance of the recording medium to be equal to or greater than 60 percent, the molar absorbance coefficient of the polymerizable compound is desirably equal to or lower than 200 mole-1-cm⁻¹. As set forth above, the compounds denoted by general formula (I) are suitably employed as recording monomers in a holographic recording medium employing a light source with a wavelength of around 400 nm since they can achieve the above-described desirable absorption characteristics.

The holographic recording medium of the present invention comprises the above recording layer (holographic recording layer), and preferably comprises a lower substrate, a filter layer, a holographic recording layer, and an upper substrate. As needed, it may comprise additional layers such as a reflective layer, filter layer, first gap layer, and second gap layer.

The holographic recording medium of the present invention is capable of recording and reproducing information through utilization of the principle of the hologram. This may be a relatively thin planar hologram that records two-dimensional information or the like, or a volumetric hologram that records large quantities of information, such as three-dimensional images. It may be either of the transmitting or reflecting type. Since the holographic recording medium of the present invention is capable of recording high volumes of information, it is suitable for use as a volume holographic recording medium of which high recording density is demanded.

The method of recording a hologram on the holographic recording medium of the present invention is not specifically limited; examples are amplitude holograms, phase holograms, blazed holograms, and complex amplitude holograms. Among these, a preferred method is the so-called “collinear method” in which recording of information in volume holographic recording regions is carried out by irradiating an informing light and a reference light onto a volume holographic recording area as coaxial beams to record information by means of interference pattern through interference of the informing light and the reference light.

Details of substrates and various layers that can be incorporated into the holographic recording medium of the present invention will be described below.

### —Substrate—

The substrate is not specifically limited in terms of its shape, structure, size, or the like; these may be suitably selected based on the objective. For example, the substrate may be disk-shaped, card-shaped, or the like. A substrate of a material capable of ensuring the mechanical strength of the holographic recording medium can be suitably selected. When the light employed for recording and reproducing enters after passing through the substrate, a substrate that is adequately transparent at the wavelength region of the light employed is desirable.

Normally, glass, ceramic, resin, or the like is employed as the substrate material. From the perspectives of moldability and cost, resin is particularly suitable. Examples of such resins are: polycarbonate resin, acrylic resin, epoxy resin, polystyrene resin, acrylonitrile-styrene copolymers, polyethylene resin, polypropylene resin, silicone resin, fluorine resin, ABS resin, and urethane resin. Of these, from the perspective of moldability, optical characteristics, and cost, polycarbonate resin and acrylic resin are preferred. Synthesized resins and commercially available resins may both be employed as substrates.

Normally, address servo areas are provided on the substrate at prescribed angular intervals as multiple positioning areas extending linearly in a radial direction, with the fan-shaped intervals between adjacent address servo areas serving as data areas. Information for operating focus servos and tracking servos by the sampled servo method, as well as address information, is recorded (reformatted) as pre-embossed pits (servo pits) or the like in address servo areas. Focus servo operation can be conducted using the reflective surface of a reflective film. Wobble pits, for example, can be employed as information for operating a tracking servo. When the holographic recording medium is card-shaped, it is possible not to have a servo pit pattern.

The thickness of the substrate is not specifically limited, and may be suitably selected based on the objective: a thickness of 0.1 to 5 mm is preferable, with 0.3 to 2 mm being preferred. A substrate thickness of equal to or greater than 0.1 mm is capable of preventing shape deformation during disk storage, while a thickness of equal to or less than 5 mm can avoid an overall disk weight generating an excessive load on the drive motor.
—Recording Layer—

The recording layer can be formed with the optical recording composition of the present invention and is capable of recording information by holography. The thickness of the recording layer is not specifically limited, and may be suitably selected based on the objective. A recording layer thickness falling within a range of 1 to 1,000 micrometers yields an adequate S/N ratio even when conducting 10 to 300 shift multiplexing, and a thickness falling within a range of 100 to 700 micrometers is advantageous in that it yields a markedly good S/N ratio.

### —Reflective Film—

A reflective film can be formed on the servo pit pattern surface of the substrate.

A material having high reflectance for the informing light and reference light is preferably employed as the material of the reflective film. When the wavelength of the light employed as the informing light and reference light ranges from 400 to 780 nm, examples of desirable materials are Al, Al alloys, Ag, and Ag alloys. When the wavelength of the light employed as the informing light and reference light is equal to or greater than 650 nm, examples of desirable materials are Al, Al alloys, Ag, Ag alloys, Au, Cu alloys, and TiN.

By employing an optical recording medium that reflects light as well as can be recorded and/or erased information such as a DVD (digital video disk) as a reflective film, it is possible to record and rewrite directory information, such as the areas in which holograms have been recorded, when rewriting was conducted, and the areas in which errors are present and for which alternate processing has been conducted, without affecting the hologram.

The method of forming the reflective film is not specifically limited and may be suitably selected based on the objective. Various vapor phase growth methods such as vacuum deposition, sputtering, plasma CVD, optical CVD, ion plating, and electron beam vapor deposition may be employed. Of these, sputtering is superior from the perspectives of mass production, film quality, and the like.

The thickness of the reflective film is preferably equal to or greater than 50 nm, more preferably equal to or greater than 100 nm, to obtain adequate reflectance.

### —Filter Layer—

A filter layer can be provided on the servo pits of the substrate, on the reflective layer, or on the first gap layer, described further below.

The filter layer has a function of reflecting selective wavelengths in which, among multiple light rays, only light of a specific wavelength is selectively reflected, permitting passing one light and reflecting a second light. It also has a function of preventing generation of noise in which irregular reflection of the informing light and the reference light by the reflective film of the recording medium is prevented without a shift in the selectively reflected wavelength even when the angle of incidence varies. Therefore, by stacking filter layers on the recording medium, it is possible to perform optical recording with high resolution and good diffraction efficiency.

The filter layer is not specifically limited and may be suitably selected based on the objective. For example, the filter layer can be comprised of a laminate in which at least one of a dichroic mirror layer, coloring material-containing layer, dielectric vapor deposition layer, single-layer or two- or more layer cholesteric layer and other layers suitably selected as needed is laminated. The thickness of the filter layer is not specifically limited and may be, for example, about 0.5 to 20 micrometers.

The filter layer may be laminated by direct application on the substrate or the like with the recording layer, or may be laminated on a base material such as a film to prepare a filter layer which is then laminated on the substrate.
—First Gap Layer—

The first gap layer is formed as needed between the filter layer and the reflective film to flatten the surface of the lower substrate. It is also effective for adjusting the size of the hologram that is formed in the recording layer. That is, since the recording layer should form a certain size of the interference region of the recording-use reference light and the informing light, it is effective to provide a gap between the recording layer and the servo pit pattern.

For example, the first gap layer can be formed by applying a material such as an ultraviolet radiation-curing resin from above the servo pit pattern and curing it. When employing a filter layer formed by application on a transparent base material, the transparent base material can serve as the first gap layer.

The thickness of the first gap layer is not specifically limited, and can be suitably selected based on the objective. A thickness of 1 to 200 micrometers is desirable.

### —Second Gap Layer—

The second gap layer is provided as needed between the recording layer and the filter layer.

The material of the second gap layer is not specifically limited, and may be suitably selected based on the objective. Examples are: transparent resin films such as triacetyl cellulose (TAC), polycarbonate (PC), polyethylene terephthalate (PET), polystyrene (PS), polysulfone (PSF), polyvinylalcohol (PVA), and poly(methyl methacrylate) (PMMA); and norbornene resin films such as a product called ARTON film made by JSR Corporation and a product called Zeonoa made by Japan Zeon Co. Of these, those that are highly isotropic are desirable, with TAC, PC, the product called ARTON, and the product called Zeonoa being preferred. The thickness of the second gap layer is not specifically limited and may be suitably selected based on the objective. A thickness of 1 to 200 micrometers is desirable.

Specific embodiments of the holographic recording medium of the present invention will be described in greater detail below. However, the present invention is not limited to these specific embodiments.

### First Implementation Embodiment

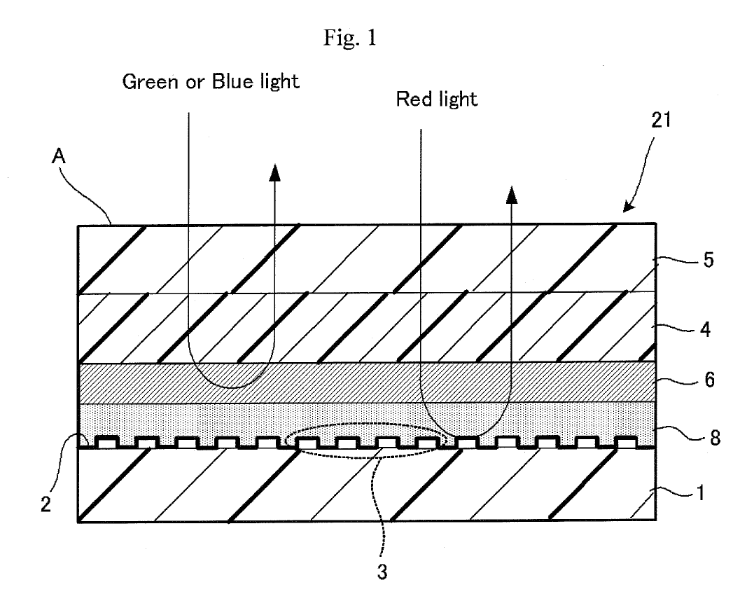

FIG. 1 is a schematic cross-sectional view of the configuration of the holographic recording medium according to the first implementation embodiment. In holographic recording medium 21 according to the first implementation embodiment, a servo pit pattern 3 is formed on substrate 1 made of polycarbonate resin or glass, and aluminum, gold, platinum, or the like is coated on servo pit pattern 3 to provide reflective film 2. In FIG. 1, servo pit pattern 3 has been formed over the entire surface of lower substrate 1, but the servo pit pattern may be formed cyclically. Servo pit pattern 3 is normally 1,750 Angstroms (175 nm) in height, and is quite small relative to the thickness of the substrate and the other layers.

First gap layer 8 is formed by spin coating or the like a material such as an ultraviolet radiation-curing resin on reflective film 2 of lower substrate 1. First gap layer 8 is effective for both the protection of reflective layer 2 and the adjustment of the size of the hologram formed in recording layer 4. That is, providing a gap between recording layer 4 and servo pit pattern 3 is effective for the formation of an interference area for the recording-use reference light and informing light of a certain size in recording layer 4.

Filter layer 6 is provided on first gap layer 8. Recording layer 4 is sandwiched between filter layer 6 and upper substrate 5 (a polycarbonate resin substrate or glass substrate) to form holographic recording medium 21.

FIG. 1 shows a filter layer 6 that passes only infrared radiation and blocks light of all other colors. Accordingly, since the informing light and recording and reproducing-use reference light are blue, they are blocked by filter layer 6 and do not reach reflective film 2. They return, exiting from entry and exit surface A.

Filter layer 6 is a multilayered vapor deposition film comprised of high refractive index layers and low refractive index layers deposited in alternating fashion.

Filter layer 6, comprised of a multilayered vapor deposition film, may be formed directly on first gap layer 8 by vacuum vapor deposition, or a film comprised of a multilayered vapor deposition film formed on a base material may be punched into the shape of a holographic recording medium to employed as filter layer 6.

In this embodiment, holographic recording medium 21 may be disk-shaped or card-shaped. When card-shaped, the servo pit pattern may be absent. In holographic recording medium 21, the lower substrate is 0.6 mm, first gap layer 8 is 100 micrometers, filter layer 6 is 2 to 3 micrometers, recording layer 4 is 0.6 mm, and upper substrate 5 is 0.6 mm in thickness, for a total thickness of about 1.9 mm.

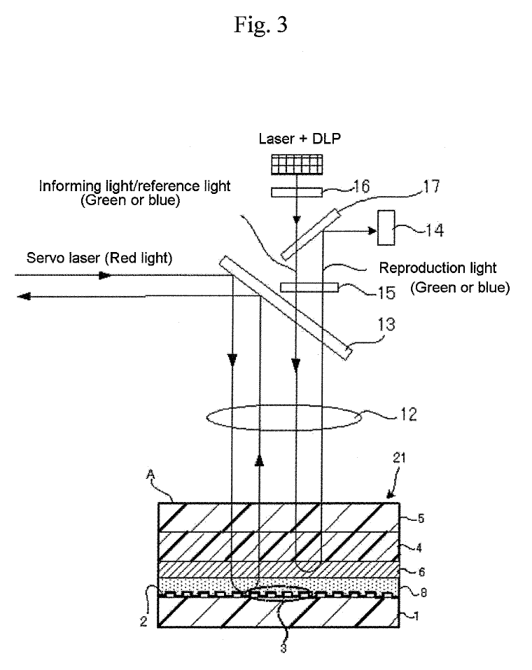

An optical system applicable for the recording of information on and the reproduction of information from holographic recording medium 21 will be described with reference to FIG. 3.

First, a light (red light) emitted by a servo laser is nearly 100 percent reflected by dichroic mirror 13, passing through objective lens 12. Objective lens 12 directs the servo light onto holographic recording medium 21 so that it focuses at a point on reflective film 2. That is, dichroic mirror 13 passes light of green and blue wavelengths while reflecting nearly 100 percent of red light. The servo light entering entry and exit surface A to which and from which the light enters and exits of holographic recording medium 21 passes through upper substrate 5, recording layer 4, filter layer 6, and first gap layer 8, is reflected by reflective layer 2, and passes back through first gap layer 8, filter layer 6, recording layer 4, and upper substrate 5, exiting entry and exit surface A. The returning light that exits passes through objective lens 12, is nearly 100 percent reflected by dichroic mirror 13, and the servo information is detected by a servo information detector (not shown in FIG. 3). The servo information that is detected is employed for focus servo, tracking servo, slide servo, and the like. When the hologram material included in recording layer 4 is not sensitive to red light, the servo light passes through recording layer 4 without affecting recording layer 4, even when the servo light is randomly reflected by reflective film 2. Since the light in the form of the servo light reflected by reflective film 2 is nearly 100 percent reflected by dichroic mirror 13, the servo light is not detected by a CMOS sensor or CCD 14 for reproduction image detection and thus does not constitute noise to the reproduction light.

The informing light and recording-use reference light generated by the recording/reproducing laser passes through polarizing plate 16 and is linearly polarized. It then passes through half mirror 17, becoming circularly polarized light at the point where it passes through ¼ wavelength plate 15. The light then passes through dichroic mirror 13, and is directed by objective lens 12 onto holographic recording medium 21 so that the informing light and recording-use reference light form an interference pattern in recording layer 4. The informing light and recording-use reference light enter through entry and exit surface A, interfering with each other to form an interference pattern in recording layer 4. Subsequently, the informing light and recording-use reference light pass through recording layer 4, entering filter layer 6. However, they are reflected before reaching the bottom surface of filter layer 6, returning. That is, neither the informing light nor the recording-use reference light reaches reflective film 2. That is because filter layer 6 is a multilayered vapor deposition layer in which multiple high refractive index and low refractive index layers are alternatively laminated, and has the property of passing only red light.

### Second Implementation Embodiment

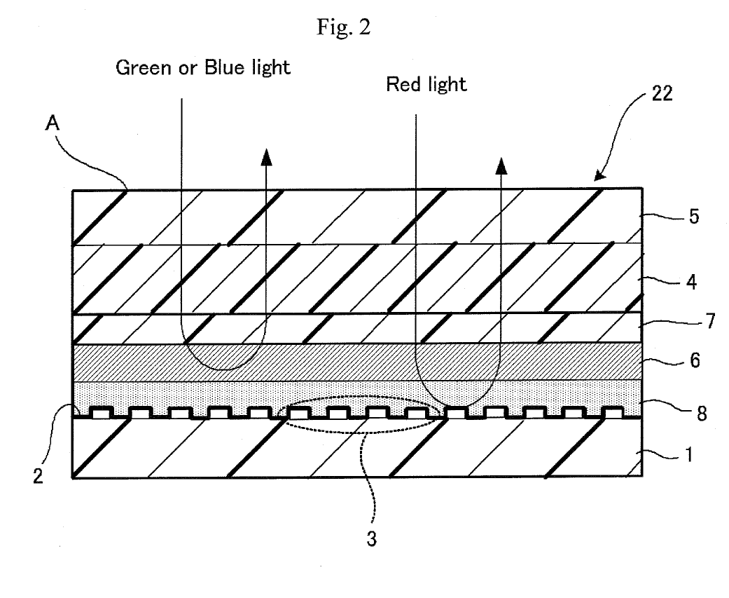

FIG. 2 is a schematic cross-sectional view of the configuration of the holographic recording medium according to the second implementation embodiment. A servo pit pattern 3 is formed on substrate 1 made of polycarbonate resin or glass in the holographic recording medium 22 according to the second implementation embodiment. Reflective film 2 is provided by coating aluminum, gold, platinum, or the like on the surface of servo pit pattern 3. Servo pit pattern 3 is normally 1,750 Angstroms (175 nm) in height in the same manner as in the first implementation embodiment.

The configuration of the second implementation embodiment differs from that of the first implementation embodiment in that second gap layer 7 is provided between filter layer 6 and recording layer 4 in holographic recording medium 22 according to the second implementation embodiment. A point at which the informing light and reproduction light are focused is present in second gap layer 7. When this area is embedded in a photopolymer, excessive consumption of monomer occurs due to excess exposure, and multiplexing recording capability diminishes. Accordingly, it is effective to provide a nonreactive transparent second gap layer.

Filter layer 6 in the form of a multilayered vapor deposition film comprised of multiple layers in which multiple high refractive index and low refractive index layers are alternately laminated is formed over first gap layer 8 once first gap layer 8 has been formed, and the same one as employed in the first implementation embodiment can be employed as filter layer 6 in the second implementation embodiment.

In holographic recording medium 22 of the second implementation embodiment, lower substrate 1 is 1.0 mm, first gap 8 is 100 micrometers, filter layer 6 is 3 to 5 micrometers, second gap layer 7 is 70 micrometers, recording layer 4 is 0.6 mm, and upper substrate 5 is 0.4 mm in thickness, for a total thickness of about 2.2 mm.

When recording or reproducing information, a red servo light and a green informing light and recording/reproducing reference light are directed onto holographic recording medium 22 of the second implementation embodiment having the configuration set forth above. The servo light enters through entry and exit surface A, passing through recording layer 4, second gap layer 7, filter layer 6, and first gap layer 8, and is reflected by reflective film 2, returning. The returning light then passes sequentially back through first gap layer 8, filter layer 6, second gap layer 7, recording layer 4, and upper substrate 5, exiting through entry and exit surface A. The returning light that exits is used for focus servo, tracking servo, and the like. When the hologram material included in recording layer 4 is not sensitive to red light, the servo light passes through recording layer 4 and is randomly reflected by reflective film 2 without affecting recording layer 4. The green informing light and the like enters through entry and exit surface A, passing through recording layer 4 and second gap layer 7, and is reflected by filter layer 6, returning. The returning light then passes sequentially back through second gap layer 7, recording layer 4, and upper substrate 5, exiting through entry and exit layer A. During reproduction, as well, the reproduction-use reference light and the reproduction light generated by irradiating the reproduction-use reference light onto recording layer 4 exit through entry and exit surface A without reaching reflective film 2. The optical action around holographic recording medium 22 (objective lens 12, filter layer 6, and detectors in the form of CMOS sensors or CCD 14 in FIG. 3) is identical to that in the first implementation embodiment and thus the description thereof is omitted.

The method of recording information on the holographic recording medium of the present invention will be described below.

An interference image can be formed on the recording layer of the holographic recording medium of the present invention by irradiation of an informing light and a reference light to the recording layer, and a fixing light can be irradiated to the recording layer on which the interference image has been formed to fix the interference image.

A light having coherent properties can be employed as the informing light. By irradiating the informing light and reference light onto the recording medium so that the optical axes of the informing light and reference light are coaxial, it is possible to record in the recording layer an interference image generated by interference of the informing light and reference light. Specifically, a informing light imparted with a two dimensional intensity distribution and a reference light of intensity nearly identical to that of the informing light are superposed in the recording layer and the interference pattern that they form is used to generate an optical characteristic distribution in the recording layer, thereby recording information. The wavelengths of the informing light and reference light are preferably equal to or greater than 400 nm, more preferably 400 to 2,000 nm, and further preferably, 400 to 700 nm.

After recording information (forming an interference image) by irradiating the informing light and reference light, a fixing light can be irradiated to fix the interference image. The wavelength of the fixing light is preferably less than 400 nm, more preferably equal to or greater than 100 nm but less than 400 nm, and further preferably, equal to or greater than 200 nm but less than 400 nm.

Information can be reproduced by irradiating a reference light onto an interference image formed by the above-described method. In the course of reading (reproducing) information that has been written, just a reference light is irradiated onto the recording layer with the same arrangement as during recording, causing a reproduction light having an intensity distribution corresponding to the optical characteristic distribution formed in the recording layer to exit the recording layer.

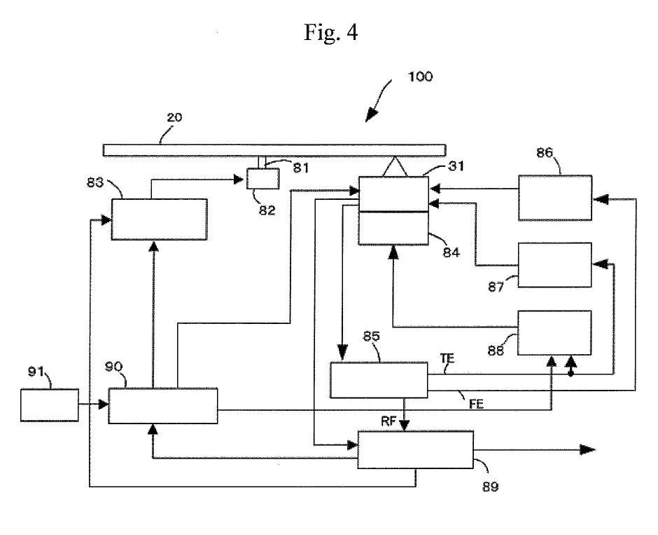

An optical recording and reproducing device suited to use in the recording and reproducing of information in the holographic recording medium of the present invention will be described with reference to FIG. 4. The optical recording and reproducing device 100 of FIG. 4 is equipped with spindle 81 on which is mounted holographic recording medium 20, spindle motor 82 rotating spindle 81, and spindle servo circuit 83 controlling spindle motor 82 so that it maintains holographic recording medium 20 at a prescribed rpm level.

Recording and reproducing device 100 is further equipped with pickup 31 for recording information by irradiating a informing light and a recording-use reference light onto holographic recording medium 20, and for reproducing information that has been recorded on holographic recording medium 20 by irradiating a reproducing-use reference light onto holographic recording medium 20 and detecting the reproduction light; and driving device 84 capable of moving pickup 31 radially with respect to holographic recording medium 20.

Optical recording and reproducing device 100 is equipped with detection circuit 85 for detecting focus error signal FE, tracking error signal TE, and reproduction signal RF based on the output signals of pickup 31; focus servo circuit 86 that operates a focus servo by driving an actuator in pickup 31 to move an objective lens (not shown in FIG. 4) in the direction of thickness of holographic recording medium 20 based on focus error signal FE detected by detection circuit 85; tracking servo circuit 87 that operates a tracking servo by driving an actuator in pickup 31 to move an objective lens in the radial direction of holographic recording medium 20 based on tracking error signal TE detected by detection circuit 85; and slide servo circuit 87 that operates a slide servo by controlling drive device 84 to move pickup 31 in the radial direction of holographic recording medium 20 based on instructions from a controller, described further below, and tracking error signal TE.

Optical recording and reproducing device 100 is further equipped with signal processing circuit 89 that decodes the output data of a CCD array or CMOS in pickup 31 to reproduce data recorded in the data areas of holographic recording medium 20, reproduces a base clock based on reproduction signal RF from detection circuit 85, and determines addresses; controller 90 that effects overall control of optical recording and reproducing device 100; and operation element 91 providing various instructions to controller 90. Controller 90 inputs the base clock and address information outputted by signal processing circuit 89 and controls pickup 31, spindle servo circuit 83, slide servo circuit 88, and the like. Spindle servo circuit 83 inputs the base clock that is outputted by signal processing circuit 89. Controller 90 comprises a central processing unit (CPU), read only memory (ROM), and random access memory (RAM). The functions of controller 90 can be realized by having the CPU that employs the RAM as a work area and execute programs stored in the ROM.

### EXAMPLES

The present invention will be described in detail below based on examples. However, the present invention is not limited to the examples.

—Examples of Synthesizing Compounds Denoted by General Formula (II)—

Example Compounds (I-2), (I-3), (I-8), and (I-9) were synthesized by the general scheme given below based on the method described in DE2830927A1. In the following scheme, R²¹¹ to R²¹³ have the same definitions as in general formula (II). Various compounds in which R²¹¹ to R²¹³ vary can be synthesized by the following scheme by employing different starting materials in synthesis.

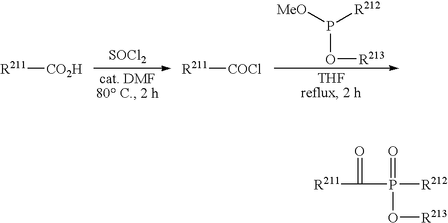

The identification results of Example Compounds (I-2), (I-3), (I-8) and (I-9) thus obtained are given below.

<I-2>

¹H NMR (300 MHz, CDCl₃) δ1.32 (t, 3H), 3.62(s, 6H), 4.13-4.26 (m, 2H), 6.49 (d, 2H), 7.32(t, 1H), 7.40-7.51 (m, 2H), 7.54-7.59(m, 1H), 7.79 (dd, 2H)

<I-3>

¹H NMR (300 MHz, CDCl₃) δ1.37 (d, 3H), 1.39 (d, 3H), 4.91-4.98 (m, 1H), 7.29 (s, 3H), 7.47-7.51 (m, 2H), 7.59-7.61 (m 1H), 7.91 (dd, 2H)

<I-8>

¹H NMR (300 MHz, CDCl₃) δ1.34 (d, 3H), 1.38 (d, 3H), 3.67(s, 6H), 4.68-4.80 (m, 1H), 7.32 (t, 1H), 7.41-7.50 (m, 2H), 7.52-7.59 (m, 1H), 7.90 (dd, 2H)

<I-9>

¹H NMR (300 MHz, CDCl₃) δ1.36 (t, 3H), 4.41 (q, 2H), 7.28 (s, 3H), 7.58-7.64 (m, 1H), 7.93 (dd, 2H)

### Absorption Characteristics of Compounds

The absorption spectra of solutions (solvent: methylene chloride; solution concentration: 1×10⁻⁵ mol/L) of Example Compounds SM-2, SM-8, SM-15, and SM-23, and Comparative Compounds 1 to 5 below were measured with a UV-3600 (made by Shimadzu Corporation), and the molar absorbance coefficient (ε) was calculated from the measured absorbance values thus obtained. The results are given in Table 1.

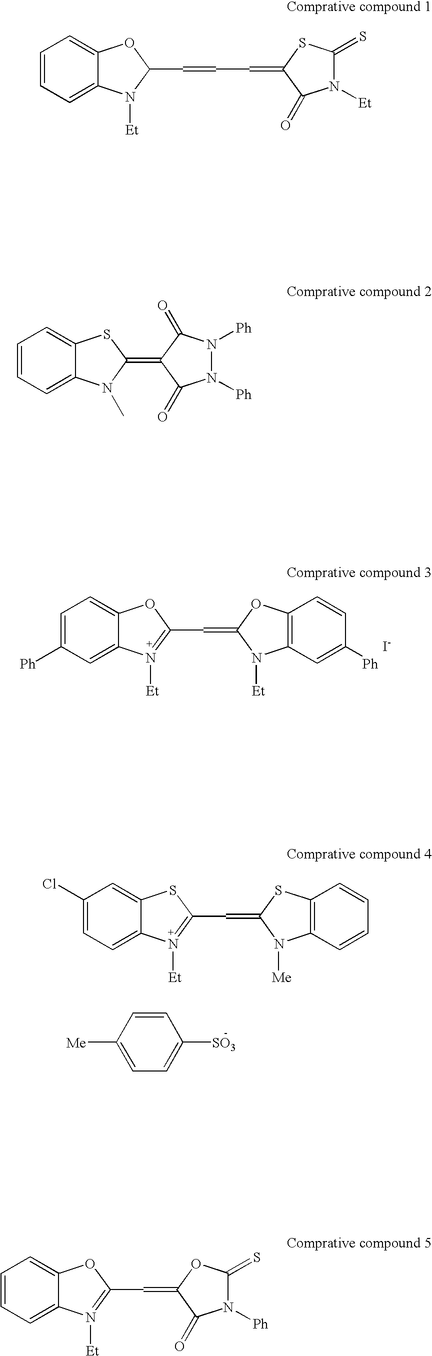

Comparative compound 1 is a dye moiety of Compound DM-14 described in Japanese Unexamined Patent Publication (KOKAI) No. 2005-275158. Comparative compound 2 is a dye moiety of Compound No. 1-1 described in Japanese Unexamined Patent Publication (KOKAI) No. 2007-272044. Comparative compound 3 is a dye moiety of Compound No. 1-2 described in Japanese Unexamined Patent Publication (KOKAI) No. 2007-272044. Comparative compound 4 is a dye moiety of Compound No. 1-3 described in Japanese Unexamined Patent Publication (KOKAI) No. 2007-272044. Comparative compound 5 is a dye moiety of Compound No. 1-5 described in Japanese Unexamined Patent Publication (KOKAI) No. 2007-272044.

TABLE 1

| | | ε at λmax | |
|---|---|---|---|
| | | (mole · l · | ε at 405 nm |
| | λmax | cm⁻¹) | (mole · l · cm⁻¹) |
| Example Compound SM-2 | 353 nm | 55,300 | ~0 |
| Example Compound SM-8 | 340 nm | 40,000 | ~0 |
| Example Compound SM-15 | 330 nm | 41,000 | 49 |
| Example Compound SM-23 | 346 nm | 58,300 | ~0 |
| Comparative Compound 1 | 492 nm | 98700 | 2260 |
| Comparative Compound 2 | 378 nm | 43900 | 7700 |
| Comparative Compound 3 | 387 nm | 100100 | 2940 |
| Comparative Compound 4 | 378 nm | 49800 | 570 |
| Comparative Compound 5 | 368 nm | 36200 | 300 |

As described in Table 1, the value of ε at 405 nm was equal to or lower than 200 mole-1-cm⁻¹, that there was almost no absorption, and that λmax was less than 405 nm for Example Compounds SM-2, SM-8, SM-15, and SM23. They were thus suitable for recording and reproduction with a laser in the 405 nm region.

### Example 1

### Preparation of Holographic Recording Composition

A 6.4 g quantity of hexamethylene diisocyanate (made by Mitsui Chemicals Polyurethanes, Inc.; trade name: Takenate T-700), 5.21 g of polypropylene oxide triol (made by Mitsui Chemicals Polyurethanes, Inc.; trade name: MN-300), 4.64 g of polyethylene glycol (made by Tokyo Chemical Industry Co., Ltd.), 1.85 g of Example Compound SM-2, 0.16 g of photo-induced polymerization initiator (2,4,6-trimethylbenzoylphenylphosphinic acid ethyl ester; trade name: Lucirin TPO-L, made by BASF Japan), and 0.20 g of amine curing catalyst (made by SAN-APRO; trade name: U-CAT 410) were mixed under a nitrogen gas flow to prepare a holographic recording composition.

### Example 2

### Preparation of Holographic Recording Composition

With the exception that Example Compound SM-2 in Example 1 was replaced with 1.85 g of Example Compound SM-8, a holographic recording composition was prepared in the same manner as in Example 1.

### Example 3

### Preparation of Holographic Recording Composition

With the exception that Example Compound SM-2 in Example 1 was replaced with 1.85 g of Example Compound SM-15, a holographic recording composition was prepared in the same manner as in Example 1.

### Example 4

### Preparation of Holographic Recording Composition

With the exception that Example Compound SM-2 in Example 1 was replaced with 1.85 g of Example Compound SM-23, a holographic recording composition was prepared in the same manner as in Example 1.

### Example 5

### Preparation of Holographic Recording Composition

With the exception that the 0.16 g of photo-induced polymerization initiator (2,4,6-trimethylbenzoylphenylphosphinic acid ethyl ester; trade name: Lucirin TPO-L, made by BASF Japan) in Example 1 was replaced with 0.16 g of Example Compound I-8, a holographic recording composition was prepared in the same manner as in Example 1.

### Example 6

### Preparation of Holographic Recording Composition

With the exception that the 0.16 g of photo-induced polymerization initiator (2,4,6-trimethylbenzoylphenylphosphinic acid ethyl ester; trade name: Lucirin TPO-L, made by BASF Japan) in Example 2 was replaced with 0.16 g of Example Compound I-8, a holographic recording composition was prepared in the same manner as in Example 2.

### Example 7

### Preparation of Holographic Recording Composition

With the exception that the 0.16 g of photo-induced polymerization initiator (2,4,6-trimethylbenzoylphenylphosphinic acid ethyl ester; trade name: Lucirin TPO-L, made by BASF Japan) in Example 3 was replaced with 0.16 g of Example Compound I-8, a holographic recording composition was prepared in the same manner as in Example 3.

### Example 8

### Preparation of Holographic Recording Composition

With the exception that the 0.16 g of photo-induced polymerization initiator (2,4,6-trimethylbenzoylphenylphosphinic acid ethyl ester; trade name: Lucirin TPO-L, made by BASF Japan) in Example 4 was replaced with 0.16 g of Example Compound I-8 (synthesized product), a holographic recording composition was prepared in the same manner as in Example 4.

### Comparative Example 1

### Preparation of Holographic Recording Composition

A 6.4 g quantity of hexamethylene diisocyanate (made by Mitsui Chemicals Polyurethanes, Inc.; trade name: Takenate T-700), 5.21 g of polypropylene oxide triol (made by Mitsui Chemicals Polyurethanes, Inc.; trade name: MN-300), 4.64 g of polyethylene glycol (made by Tokyo Chemical Industry Co., Ltd.), 1.85 g of 2,4,6-tribromophenyl acrylate (Dai-ichi Kogyo Seiyaku Co., Ltd.; trade name BR-30), 0.16 g of photo-induced polymerization initiator (2,4,6-trimethylbenzoylphenylphosphinic acid ethyl ester; trade name: Lucirin TPO-L, made by BASF Japan), and 0.20 g of amine curing catalyst (made by SAN-APRO; trade name: U-CAT 410) were mixed under a nitrogen gas flow to prepare a holographic recording composition.

### Comparative Example 2

### Preparation of Holographic Recording Composition

With the exception that the 1.85 g of 2,4,6-tribromophenyl acrylate (Dai-ichi Kogyo Seiyaku Co., Ltd.; trade name BR-30) employed in Comparative Example 1 was replaced with 1.85 g of the following monomer (R-1) (λmax=426 nm (ε=53100) in CH₂Cl₂, ε_{at 405 nm}=36100) described in Japanese Unexamined Patent Publication (KOKAI) No. 2005-275158, a holographic recording composition was prepared in the same manner as in Comparative Example 1.

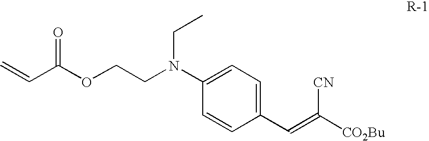

### Comparative Example 3

### Preparation of Holographic Recording Composition

With the exception that the 1.85 g of 2,4,6-tribromophenyl acrylate (Dai-ichi Kogyo Seiyaku Co., Ltd.; trade name BR-30) employed in Comparative Example 1 was replaced with 1.85 g of the following monomer (R-2) (λmax=460 nm (ε=68000) in CH₂Cl₂, ε_{at 405 nm}=45200) described in Japanese Unexamined Patent Publication (KOKAI) No. 2007-272044, a holographic recording composition was prepared in the same manner as in Comparative Example 1.

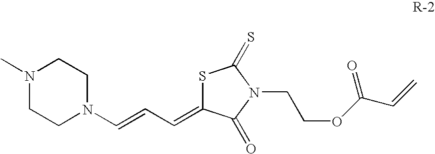

### Examples 9 to 16 and Comparative Examples 4 to 6

### Preparation of Optical Recording Media

A first substrate was prepared by subjecting one side of a glass sheet 0.5 mm in thickness to an antireflective treatment to impart a reflectance of 0.1 percent for perpendicularly incident light with the wavelength of 405 nm.

A second substrate was prepared by subjecting one side of a glass sheet 0.5 mm in thickness to an aluminum vapor deposition treatment to impart a reflectance of 90 percent for perpendicularly incident light with the wavelength of 405 nm.

A transparent polyethylene terephthalate sheet 500 micrometers in thickness was provided as a spacer on the side of the first substrate that had not been subjected to the antireflective treatment.

The holographic recording compositions of Examples 1 to 4 and Comparative Examples 1 to 8 were each separately placed on first substrates, the aluminum vapor deposited surface of the second substrates were stacked on the holographic recording composition in such a manner that air was not entrained, and the first and second substrates were bonded through the spacer. Subsequently, Examples 9 to 16 and Comparative Examples 4 to 6 were left for 6 hours at 80° C. to prepare various optical recording media (holographic recording media). The thickness of the recording layers formed was 200 micrometers in all media prepared.

### <Recording in the Optical Recording Medium and Evaluation

(1)Measurement of Recording Sensitivity

Employing a hologram recording and reproduction tester, a series of multiplexed holograms was written into the various optical recording media that had been prepared at a spot recording diameter of 200 micrometers at the focal position of the recording hologram, and the sensitivity (recording energy) was measured as follows.

### —Sensitivity Measurement—

The beam energy during recording (mJ/cm²) was varied and the change in the error rate (BER: bit error rate) of the reproduced signal was measured. Normally, there is such a tendency that the luminance of the reproduced signal increases and the BER of the reproduced signal gradually drops with an increase in the irradiated light energy. In the measurement, the lowest light energy at which a fairly good reproduced image (BER<10⁻³) was obtained was adopted as the recording sensitivity of the holographic recording medium. The wavelength of the informing light and reference light for recording as well as the wavelength of the reproduction light were 405 nm.

(2) Measurement of Recording Capacity by Planar Wave Tester

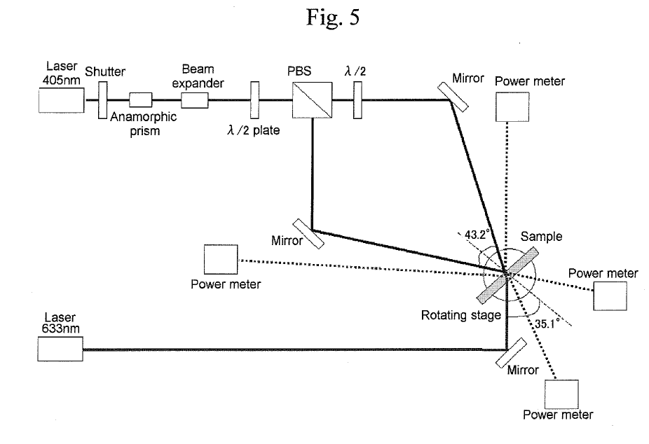

FIG. 5 shows a schematic of the optical system of a planar wave recording tester. A “Littrow” blue laser made by SONY (wavelength: 405 nm) was employed as the recording light source and an He—Ne laser (wavelength: 633 nm) that was not absorbed by the medium was employed as the probe light source. The luminous energy of the recording light source was 4 [mW] with the informing light and reference light combined. The luminous energy of the probe light source was 5 [mW]. The crossing angle of the informing light and the reference light was 43.2° (grating interval: 550 nm). The angle of incidence of the probe light—the angle at which the Bragg condition was satisfied—was 35.1°. A recording spot diameter of 6 mm was employed. The dynamic range of the storage capacity is denoted by an index referred to as “M#”. The recording capacity of each of the optical recording media of Example 9 to 16 and Comparative Examples 4 to 6 was measured with the above-described optical system. The measurement is described below.

Adopting a diffraction efficiency of 1 to 3 percent per cycle as standard, in a manner not exceeding 10 percent, 61 multiplexed recordings were conducted at intervals of 1° from −30° to +30° until the sensitivity of the recording material almost disappeared. Fixing was conducted until absorption of the recording light source by the sample almost ceased (fixing light source: High-power UV-LED (UV-400) made by Keyence), the angular selectivity was evaluated at 0.01° intervals from −32° to +32°, and the square roots of the diffraction efficiencies ηᵢ of the peaks obtained were summed to calculate M#. Diffraction efficiency η was evaluated as set forth below. The results are given in Table 2.

η=diffracted light/(diffracted light+transmitted light)×100 M#=Σ√ηᵢ

(3) Measurement of Transmittance T

The transmittance at a wavelength of 405 nm was measured with a UV-3600 (made by Shimadzu Corporation) for each of the optical recording media prepared in Examples 9 to 16 and Comparative Examples 4 to 6.

(4) Molar Absorbance Coefficient

Each of the recording monomers contained in the holographic recording compositions prepared in Examples 1 to 8 and Comparative Examples 1 to 3 was dissolved in methylene chloride to a concentration of 5×10⁻⁵ mol/L, the absorption spectrum of each solution prepared was measured with a UV-3600 (made by Shimadzu Corporation), and the absorption at 405 nm was measured. The molar absorbance coefficient was calculated from the absorbance thus measured. The results are given in Table 2.

TABLE 2

| | | | | Transmittance | |
|---|---|---|---|---|---|
| | Holographic | Recording | Recording | T of the | ε |
| | recording | sensitivity | capacity | medium at | at 405 nm |
| | composition | (mJ/cm²) | (M#) | 405 nm (%) | (mole · l · cm⁻¹) |
| Example 9 | Example 1 | 51 | 11.2 | 85.5 | ~0 |
| Example 10 | Example 2 | 54 | 10.9 | 85.8 | ~0 |
| Example 11 | Example 3 | 55 | 10.8 | 77.6 | 49 |
| Example 12 | Example 4 | 52 | 11.0 | 85.2 | ~0 |
| Example 13 | Example 5 | 35 | 13.2 | 88.5 | ~0 |
| Example 14 | Example 6 | 42 | 12.8 | 88.7 | ~0 |
| Example 15 | Example 7 | 43 | 12.7 | 80.0 | 49 |
| Example 16 | Example 8 | 37 | 13.0 | 88.3 | ~0 |
| Comp. Ex. 4 | Comp. Ex. 1 | 80 | 9.0 | 85.3 | ~0 |
| Comp. Ex. 5 | Comp. Ex. 2 | — | — | 0.0 | 36100 |
| Comp. Ex. 6 | Comp. Ex. 3 | — | — | 0.0 | 45200 |

The results of Table 2 show that the optical recording media of Examples 9 to 16, in which the holographic recording compositions of Examples 1 to 8 were employed, all had better recording sensitivity and greater recording capacity than the optical recording media of Comparative Examples 4 to 6, in which the holographic recording compositions of Comparative Examples 1 to 3 were employed. Recording and reproduction were impossible with Comparative Examples 5 and 6, which had considerable absorption at a wavelength of 405 nm.

The optical recording composition of the present invention is capable of high density recording, and is thus suitable for use in the manufacturing of various volume hologram-type optical recording media capable of high-density image recording.

Although the present invention has been described in considerable detail with regard to certain versions thereof, other versions are possible, and alterations, permutations and equivalents of the version shown will become apparent to those skilled in the art upon a reading of the specification and study of the drawings. Also, the various features of the versions herein can be combined in various ways to provide additional versions of the present invention. Furthermore, certain terminology has been used for the purposes of descriptive clarity, and not to limit the present invention. Therefore, any appended claims should not be limited to the description of the preferred versions contained herein and should include all such alterations, permutations, and equivalents as fall within the true spirit and scope of the present invention.

Having now fully described this invention, it will be understood to those of ordinary skill in the art that the methods of the present invention can be carried out with a wide and equivalent range of conditions, formulations, and other parameters without departing from the scope of the invention or any embodiments thereof.

All patents and publications cited herein are hereby fully incorporated by reference in their entirety. The citation of any publication is for its disclosure prior to the filing date and should not be construed as an admission that such publication is prior art or that the present invention is not entitled to antedate such publication by virtue of prior invention.

Unless otherwise stated, a reference to a compound or component includes the compound or component by itself, as well as in combination with other compounds or components, such as mixtures of compounds.

As used herein, the singular forms “a,” “an,” and “the” include the plural reference unless the context clearly dictates otherwise.

Except where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not to be considered as an attempt to limit the application of the doctrine of equivalents to the scope of the claims, each numerical parameter should be construed in light of the number of significant digits and ordinary rounding conventions.

Additionally, the recitation of numerical ranges within this specification is considered to be a disclosure of all numerical values and ranges within that range. For example, if a range is from about 1 to about 50, it is deemed to include, for example, 1, 7, 34, 46.1, 23.7, or any other value or range within the range.

What is claimed is:

1. An optical recording composition comprising at least one optical recording compound selected from the group consisting of:
a compound denoted by general formula (I-1): 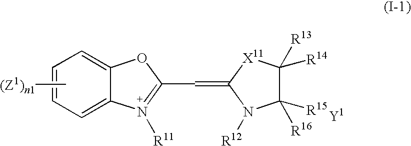
wherein, in general formula (I-1), each of R¹¹ and R¹² independently denotes an alkyl group, alkenyl group, aryl group, or heterocyclic group, each of R¹³, R¹⁴, R¹⁵, and R¹⁶ independently denotes a hydrogen atom or a substituent, X¹¹ denotes —S—, —O—, —NR¹¹¹—, or —CR¹¹²R¹¹³, each of R¹¹¹, R¹¹², and R¹¹³ independently denotes a hydrogen atom, alkyl group, alkenyl group, aryl group, or heterocyclic group, R¹¹² and R¹¹³ may bond together to form a ring structure, Z¹ denotes a substituent, n1 denotes an integer ranging from 0 to 4, plural substituents denoted by Z¹ are identical to or different from each other when n1 denotes an integer of equal to or greater than 2, Y¹ denotes an ionic species that neutralizes an electric charge of the compound denoted by general formula (I-1), and at least one from among R¹¹, R¹², R¹³, R¹⁴, R¹⁵, R¹⁶, X¹¹, and Z¹ comprises at least one polymerizable group;
a compound denoted by general formula (I-2): 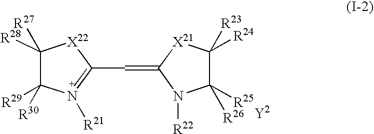
wherein, in general formula (I-2), each of R²¹ and R²² independently denotes an alkyl group, alkenyl group, aryl group, or heterocyclic group, each of R²³, R²⁴, R²⁵, R²⁶, R²⁷, R²⁸, R²⁹, and R³⁰ independently denotes a hydrogen atom or a substituent, each of X²¹ and X²² independently denotes —S—, —O—, —NR¹¹¹—, or —CR¹¹²R¹¹³—, each of R¹¹¹, R¹¹², and R¹¹³ independently denotes a hydrogen atom, alkyl group, alkenyl group, aryl group, or heterocyclic group, R¹¹² and R¹¹³ may bond together to form a ring structure, Y² denotes an ionic species that neutralizes an electric charge of the compound denoted by general formula (I-2), and at least one from among R²¹, R²², R²³, R²⁴, R²⁶, R²⁷, R²⁸, R²⁹, R³⁰, X²¹, and X²² comprises at least one polymerizable group;
a compound denoted by general formula (I-3): 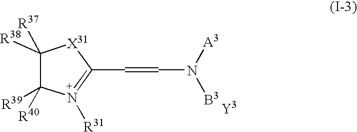
wherein, in general formula (I-3), R³¹ denotes an alkyl group, alkenyl group, aryl group, or heterocyclic group, each of R³⁷, R³⁸, R³⁹, and R⁴⁰ independently denotes a hydrogen atom or a substituent, X³¹ denotes —S—, —O—, NR¹¹¹—, or —CR¹¹²R¹¹³, each of R¹¹¹, R¹¹², and R¹¹³ independently denotes a hydrogen atom, alkyl group, alkenyl group, aryl group, or heterocyclic group, R¹¹² and R¹¹³ may bond together to form a ring structure, each of A³ and B³ independently denotes a hydrogen atom, alkyl group, alkenyl group, aryl group, heterocyclic group, or acyl group, A³ and B³ may bond together to form a ring structure, Y³ denotes an ionic species that neutralizes an electric charge of the compound denoted by general formula (I-3), and at least one from among R³¹, R³⁷, R³⁸, R³⁹, R⁴⁰, X³¹, A³, and B³ comprises at least one polymerizable group; and
a compound denoted by general formula (I-4): 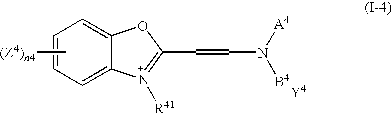
wherein, in general formula (I-4), R⁴¹ denotes an alkyl group, alkenyl group, aryl group, or heterocyclic group, each of A⁴ and B⁴ independently denotes a hydrogen atom, alkyl group, alkenyl group, aryl group, heterocyclic group, or acyl group, A⁴ and B⁴ may bond together to form a ring structure, Z⁴ denotes a substituent, n4 denotes an integer ranging from 0 to 4, plural substituents denoted by Z⁴ are identical to or different from each other when n4 denotes an integer of equal to or greater than 2, Y⁴ denotes an ionic species that neutralizes an electric charge of the compound denoted by general formula (I-4), and at least one from among R²¹, A⁴, B⁴, and Z⁴ comprises at least one polymerizable group.

2. The optical recording composition according to claim 1, wherein the optical recording compound has a molar absorbance coefficient of equal to or lower than 200 mole-1-cm⁻¹ at a wavelength of 405 nm.

3. The optical recording composition according to claim 1, wherein the optical recording compound has a maximum absorption wavelength of less than 405 nm.

4. The optical recording composition according to claim 1, wherein the polymerizable group is a radical polymerizable group.

5. The optical recording composition according to claim 1, further comprising at least one photo-induced polymerization initiator.

6. The optical recording composition according to claim 5, wherein the photo-induced polymerization initiator is a compound denoted by general formula (II): 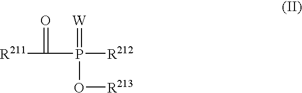
wherein, in general formula (II), each of R²¹¹, R²¹², and R²¹³ independently denotes an alkyl group, aryl group, or heterocyclic group, and W denotes an oxygen atom or sulfur atom.

7. The optical recording composition according to claim 1, further comprising at least one polyfunctional isocyanate and polyfunctional alcohol.

8. The optical recording composition according to claim 1, which is a holographic recording composition.

9. A holographic recording medium comprising a recording layer, wherein the recording layer comprises at least one optical recording compound selected from the group consisting of:
a compound denoted by general formula (I-1): 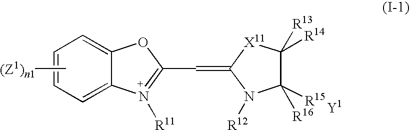
wherein, in general formula (I-1), each of R¹¹ and R¹² independently denotes an alkyl group, alkenyl group, aryl group, or heterocyclic group, each of R¹³, R¹⁴, R¹⁵, and R¹⁶ independently denotes a hydrogen atom or a substituent, X¹¹ denotes —S—, —O—, —NR¹¹¹—, or —CR¹¹²R¹¹³—, each of R¹¹¹, R¹¹², and R¹¹³ independently denotes a hydrogen atom, alkyl group, alkenyl group, aryl group, or heterocyclic group, R¹¹ and R¹¹³ may bond together to form a ring structure, Z¹ denotes a substituent, n1 denotes an integer ranging from 0 to 4, plural substituents denoted by Z¹ are identical to or different from each other when n1 denotes an integer of equal to or greater than 2, Y¹ denotes an ionic species that neutralizes an electric charge of the compound denoted by general formula (I-1), and at least one from among R¹¹, R¹², R¹³, R¹⁴, R¹⁵, R¹⁶, X¹¹, and Z¹ comprises at least one polymerizable group;
a compound denoted by general formula (I-2): 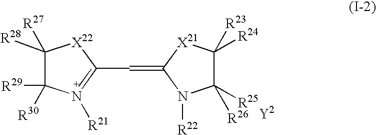
wherein, in general formula (I-2), each of R²¹ and R²² independently denotes an alkyl group, alkenyl group, aryl group, or heterocyclic group, each of R²³, R²⁴, R²⁵, R²⁶, R²⁷, R²⁸, and R³⁰ independently denotes a hydrogen atom or a substituent, each of X²¹ and X²² independently denotes —S—, —O—, —NR¹¹¹—, or —CR¹¹²R¹¹³—, each of R¹¹¹, R¹¹², and R¹¹³ independently denotes a hydrogen atom, alkyl group, alkenyl group, aryl group, or heterocyclic group, R¹¹² and R¹¹³ may bond together to form a ring structure, Y² denotes an ionic species that neutralizes an electric charge of the compound denoted by general formula (I-2), and at least one from among R²¹, R²², R²³, R²⁴, R²⁵, R²⁶, R²⁷, R²⁸, R²⁹, R³⁰, X²¹, and X²² comprises at least one polymerizable group;
a compound denoted by general formula (I-3): 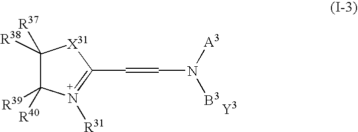
wherein, in general formula (I-3), R³¹ denotes an alkyl group, alkenyl group, aryl group, or heterocyclic group, each of R³⁷, R³⁸, R³⁹, and R⁴⁰ independently denotes a hydrogen atom or a substituent, X³¹ denotes —S—, —O—, —NR¹¹¹—, or —CR¹¹²R¹¹³, each of R¹¹¹, R¹¹², and R¹¹³ independently denotes a hydrogen atom, alkyl group, alkenyl group, aryl group, or heterocyclic group, R¹¹² and R¹¹³ may bond together to form a ring structure, each of A³ and B³ independently denotes a hydrogen atom, alkyl group, alkenyl group, aryl group, heterocyclic group, or acyl group, A³ and B³ may bond together to form a ring structure, Y³ denotes an ionic species that neutralizes an electric charge of the compound denoted by general formula (I-3), and at least one from among R³¹, R³⁷, R³⁸, R³⁹, R⁴⁰, X³¹, A³, and B³ comprises at least one polymerizable group; and
a compound denoted by general formula (I-4): 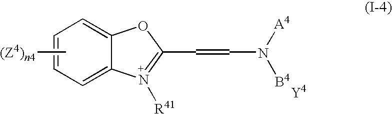
wherein, in general formula (I-4), R⁴¹ denotes an alkyl group, alkenyl group, aryl group, or heterocyclic group, each of A⁴ and B⁴ independently denotes a hydrogen atom, alkyl group, alkenyl group, aryl group, heterocyclic group, or acyl group, A⁴ and B⁴ may bond together to form a ring structure, Z⁴ denotes a substituent, n4 denotes an integer ranging from 0 to 4, plural substituents denoted by Z⁴ are identical to or different from each other when n4 denotes an integer of equal to or greater than 2, Y4 denotes an ionic species that neutralizes an electric charge of the compound denoted by general formula (I-4), and at least one from among R⁴¹, A⁴, B⁴, and Z⁴ comprises at least one polymerizable group.

10. The holographic recording medium according to claim 9, wherein the optical recording compound has a molar absorbance coefficient of equal to or lower than 200 mole-1-cm⁻¹ at a wavelength of 405 nm.

11. The holographic recording medium according to claim 9, wherein the optical recording compound has a maximum absorption wavelength of less than 405 nm.

12. The holographic recording medium according to claim 9, wherein the polymerizable group is a radical polymerizable group.

13. The holographic recording medium according to claim 9, wherein the recording layer further comprises at least one photo-induced polymerization initiator.

14. The holographic recording medium according to claim 13, wherein the photo-induced polymerization initiator is a compound denoted by general formula (II): 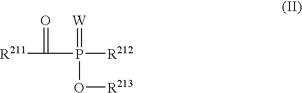
wherein, in general formula (II), each of R²¹¹, R²¹², and R²¹³ independently denotes an alkyl group, aryl group, or heterocyclic group, and W denotes an oxygen atom or sulfur atom.

15. The holographic recording medium according to claim 9, wherein the recording layer further comprises at least one polyfunctional isocyanate and polyfunctional alcohol.
